(19) United States
(12) Patent Application Publication
(10) Pub. No.: US 2022/0332845 A1
(43) Pub. Date: Oct. 20, 2022

(54) INHIBITION OF MTHFD1L FOR USE IN HYPERTROPHIC HEART DISEASE AND HEART FAILURE

(71) Applicant: GENOME BIOLOGICS UG, Kronberg im Taunus (DE)

(72) Inventors: Jaya KRISHNAN, Frankfurt am Main Hessen (DE); Corinne BISCHOF, Münsingen (CH)

(73) Assignee: GENOME BIOLOGICS UG, Kronberg im Taunus (DE)

(21) Appl. No.: 17/639,950

(22) PCT Filed: Sep. 7, 2020
(86) PCT No.: PCT/EP2020/074919

§ 371 Date: Mar. 3, 2022

(30) Foreign Application Priority Data
Sep. 5, 2019 (EP) 19195752.1
Mar. 13, 2020 (EP) 20163146.2

(51) Int. Cl. C07K 16/40; C12N 15/115; C12N 15/113; A61P 9/04

(52) U.S. Cl. CPC C07K 16/40; C12N 15/115; C12N 15/1137; A61P 9/04; C12N 2310/11; C12N 2310/14; A61K 39/00

(57) ABSTRACT

The present invention relates to a non-agonist ligand, particularly an antibody, that specifically binds to MTHFD1L or an oligonucleotide suppressing expression of MTHFD1L for use in treatment of hypertrophic heart disease and failure. The invention also relates to a method for identifying a small-molecule inhibitor of MTHFD1L.

The present invention relates to a non-agonist ligand, particularly an antibody, that specifically binds to MTHFD1L, or an oligonucleotide suppressing expression of MTHFD1L, for use in treatment of hypertrophic heart disease and heart failure. The invention also relates to a method for identifying a small-molecule inhibitor of MTHFD1L.

### BACKGROUND OF THE INVENTION

The mechanism through which stress signaling promotes endoreplication to raise the cell size threshold is unknown. While the analysis of cell cycle regulation, growth signaling pathways, and transcriptional and translational networks has provided insight into upstream control networks, the more fundamental question of the underlying energetics in which the aforementioned regulators can function remains largely unaddressed. The energetic state of a cell determines ploidy, cell size and function to establish an environment permissive for specific outputs that are co-opted by signaling cascades and transcriptional regulators to implement specific growth or functional endpoints. Emerging evidence links deregulated ADP:ATP homeostasis to the development of human hypertrophic cardiomyopathy (HCM) and aortic stenosis (AS). HCM and AS epitomize the cardiac overgrowth phenotype and are characterized by endoreplication, resulting in polyploidy and multinucleation, and pathologic cardiomyocyte growth in an environment of depressed mitochondrial ATP synthesis. It is paradoxical that energetically deficient cardiomyocytes, largely incapable of generating energy and cofactors though mitochondrial oxidation, are able to drive anabolic processes to support cardiomyocyte hypertrophy. Accordingly, mitochondrial myopathies are predominantly characterized by cardiac overgrowth.

Based on the above-mentioned state of the art, the objective of the present invention is to provide means and methods to improve the treatment of heart disease. This objective is attained by the subject-matter of the independent claims of the present specification.

### SUMMARY OF THE INVENTION

A first aspect of the invention relates to an agent for use in prevention or treatment of hypertrophic heart disease, wherein the agent is selected from
a. a non-agonist biopolymer ligand specifically binding to MTHFD1L;
b. a nucleic acid capable of specifically suppressing expression of MTHFD1L.

A second aspect of the invention relates to a nucleic acid molecule encoding the agent according to the first aspect for use in prevention or treatment of hypertrophic heart disease.

A third aspect of the invention relates to a nucleic acid expression vector comprising the nucleic acid molecule of the second aspect for use in prevention or treatment of hypertrophic heart disease.

A fourth aspect of the invention relates to a pharmaceutical composition for use in prevention or treatment of hypertrophic heart disease in a patient, comprising the agent according to the first aspect, the nucleic acid molecule according to the second aspect, or the nucleic acid vector according to the third aspect, and a pharmaceutically acceptable carrier.

A fifth aspect of the invention relates to a method for identifying an MTHFD1L inhibitor.

In another embodiment, the present invention relates a pharmaceutical composition comprising at least one of the compounds of the present invention or a pharmaceutically acceptable salt thereof and at least one pharmaceutically acceptable carrier, diluent or excipient.

### BRIEF DESCRIPTION OF THE FIGURES

FIG. 1 F₁F₀ ATP synthase controls endoreplication and multinucleation in pathologic growth.

FIG. 2 Validation of ATP5A1 downregulation and cell growth.

FIG. 3 ATP synthase inactivation promotes de novo nucleotide biosynthesis.

FIG. 4 ATP synthase controls cardiomyocyte karyokinesis and pathologic growth.

FIG. 5 MTHFD1L is necessary for multinucleation and pathologic cardiac hypertrophy and dysfunction in vivo.

FIG. 6 Validation of ectopic MTHFD1L expression in NRCs.

FIG. 7 T3 promotes purine biosynthesis and cell growth in a MIR27B-dependent manner.

FIG. 8 HIF1α-MIR27B-ATP5A1-MTHFD1L axis promotes mitosis.

FIG. 9 Validation of AAV9-fl/fl-shMthfd1l and AAV9-fl/fl-shAtp5a1 viruses and its effect on nucleation and cardiac dimension in mice.

### DETAILED DESCRIPTION OF THE INVENTION

### Terms and Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term MTHFD1L in the context of the present specification relates to monofunctional C1-tetrahydrofolate synthase, mitochondrial also known as formyltetrahydrofolate synthetase (Uniprot-ID: Q6UB35).

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

Amino acid residue sequences are given from amino to carboxyl terminus. Capital letters for sequence positions refer to L-amino acids in the one-letter code (Stryer, Biochemistry, 3rd ed. p. 21). Lower case letters for amino acid sequence positions refer to the corresponding D- or (2R)-amino acids. Sequences are written left to right in the direction from the amino to the carboxy terminus.

The terms capable of forming a hybrid or hybridizing sequence in the context of the present specification relate to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and the backbone chemistry.

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). Wherever reference is made herein to a hybridizing sequence, such hybridizing sequence may be composed of any of the above nucleotides, or mixtures thereof.

The term nucleic acid expression vector in the context of the present specification relates to a plasmid or a viral genome, which is used to transfect (in case of a plasmid) or transduce (in case of a viral genome) a target cell with a certain gene of interest. The gene of interest is under control of a promoter sequence and the promoter sequence is active inside the target cell, thus, the gene of interest is transcribed either constitutively or in response to a stimulus or dependent on the cell's status. In certain embodiments, the viral genome is packaged into a capsid to become a viral vector, which is able to transduce the target cell. In the present specification, the gene of interest encodes the agent for use in prevention or treatment of hypertrophic heart disease, wherein the agent is either a non-agonist biopolymer ligand specifically binding to MTHFD1L or a nucleic acid capable of specifically suppressing expression of MTHFD1L.

The term siRNA (small/short interfering RNA) in the context of the present specification relates to an RNA molecule capable of interfering with the expression (in other words: inhibiting or preventing the expression) of a gene comprising a nucleic acid sequence complementary or hybridizing to the sequence of the siRNA in a process termed RNA interference. The term siRNA is meant to encompass both single stranded siRNA and double stranded siRNA. siRNA is usually characterized by a length of 17-24 nucleotides. Double stranded siRNA can be derived from longer double stranded RNA molecules (dsRNA). According to prevailing theory, the longer dsRNA is cleaved by an endo-ribonuclease (called Dicer) to form double stranded siRNA. In a nucleoprotein complex (called RISC), the double stranded siRNA is unwound to form single stranded siRNA. RNA interference often works via binding of an siRNA molecule to the mRNA molecule having a complementary sequence, resulting in degradation of the mRNA. RNA interference is also possible by binding of an siRNA molecule to an intronic sequence of a pre-mRNA (an immature, non-spliced mRNA) within the nucleus of a cell, resulting in degradation of the pre-mRNA.

The term shRNA (small hairpin RNA) in the context of the present specification relates to an artificial RNA molecule with a tight hairpin turn that can be used to silence target gene expression via RNA interference (RNAi).

The term miRNA (microRNA) in the context of the present specification relates to a small non-coding RNA molecule (containing about 22 nucleotides) that functions in RNA silencing and post-transcriptional regulation of gene expression.

The term antisense oligonucleotide in the context of the present specification relates to an oligonucleotide having a sequence substantially complimentary to, and capable of hybridizing to, an RNA. Antisense action on such RNA will lead to modulation, particular inhibition or suppression of the RNA's biological effect. If the RNA is an mRNA, expression of the resulting gene product is inhibited or suppressed. Antisense oligonucleotides can consist of DNA, RNA, nucleotide analogues and/or mixtures thereof. The skilled person is aware of a variety of commercial and non-commercial sources for computation of a theoretically optimal antisense sequence to a given target. Optimization can be performed both in terms of nucleobase sequence and in terms of backbone (ribo, deoxyribo, analogue) composition. Many sources exist for delivery of the actual physical oligonucleotide, which generally is synthesized by solid state synthesis.

In the context of the present specification, the term antibody refers to whole antibodies including but not limited to immunoglobulin type G (IgG), type A (IgA), type D (IgD), type E (IgE) or type M (IgM), any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. Similarly, the term encompasses a so-called nanobody or single domain antibody, an antibody fragment consisting of a single monomeric variable antibody domain.

In the context of the present specification, the term humanized antibody refers to an antibody originally produced by immune cells of a non-human species, the protein sequences of which have been modified to increase their similarity to antibody variants produced naturally in humans. The term humanized antibody as used herein includes antibodies in which CDR sequences derived from the germline of another mammalian species, such as a mouse, have been grafted onto human framework sequences. Additional framework region modifications may be made within the human framework sequences as well as within the CDR sequences derived from the germline of another mammalian species.

The term antibody-like molecule in the context of the present specification refers to a molecule capable of specific binding to another molecule or target with high affinity/a Kd≤10E-8 mol/l. An antibody-like molecule binds to its target similarly to the specific binding of an antibody. The term antibody-like molecule encompasses a repeat protein, such as a designed ankyrin repeat protein (Molecular Partners, Zurich), an engineered antibody mimetic proteins exhibiting highly specific and high-affinity target protein binding (see US2012142611, US2016250341, US2016075767 and US2015368302, all of which are incorporated herein by reference). The term antibody-like molecule further encompasses, but is not limited to, a polypeptide derived from armadillo repeat proteins, a polypeptide derived from leucine-rich repeat proteins and a polypeptide derived from tetratricopeptide repeat proteins.

The term antibody-like molecule further encompasses a specifically binding polypeptide derived from a protein A domain, fibronectin domain FN3, consensus fibronectin domains, a lipocalins (see Skerra, Biochim. Biophys. Acta 2000, 1482(1-2):337-50), a polypeptide derived from a Zinc finger protein (see Kwan et al. Structure 2003, 11(7):803-813), Src homology domain 2 (SH2) or Src homology domain 3 (SH3), a PDZ domain, gamma-crystallin, ubiquitin, a cysteine knot polypeptide or a knottin, cystatin, Sac7d, a triple helix coiled coil (also known as alphabodies), a Kunitz domain or a Kunitz-type protease inhibitor and a carbohydrate binding module 32-2.

A viral vector in the context of the present invention refers to a vector which is derived from a virion, but the viral vector is engineered to be replication-incompetent by removing certain genes from the viral genome.

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≤10⁻⁷ mol/L when binding to its target, but a dissociation constant at least three orders of magnitude higher in its interaction with a molecule having a globally similar chemical composition as the target, but a different three-dimensional structure.

In the context of the present specification, the term dissociation constant (K_{D}) is used in its meaning known in the art of chemistry and physics; it refers to an equilibrium constant that measures the propensity of a complex composed of [mostly two] different components to dissociate reversibly into its constituent components. The complex can be e.g. an antibody-antigen complex AbAg composed of antibody Ab and antigen Ag. K_{D} is expressed in molar concentration [mol/l] and corresponds to the concentration of [Ab] at which half of the binding sites of [Ag] are occupied, in other words, the concentration of unbound [Ab] equals the concentration of the [AbAg] complex. The dissociation constant can be calculated according to the following formula:

$${K}_{D} = \frac{[ A b ] * [ A g ]}{[ A b A g ]}$$

[Ab]: concentration of antibody; [Ag]: concentration of antigen; [AbAg]: concentration of antibody antigen complex

In the context of the present specification, the terms off-rate (Koff; [1/sec]) and on-rate (Kon; [1/sec*M]) are used in their meaning known in the art of chemistry and physics; they refer to a rate constant that measures the dissociation (Koff) or association (Kon) of 5 an antibody with its target antigen. Koff and Kon can be experimentally determined using methods well established in the art.

A method for determining the Koff and Kon of an antibody employs surface plasmon resonance. This is the principle behind biosensor systems such as the Biacore® or the ProteOn® system. They can also be used to determine the dissociation constant KD by using the following formula:

$${K}_{D} = \frac{[ {K}_{off} ]}{[ {K}_{on} ]}$$

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. heart disease) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

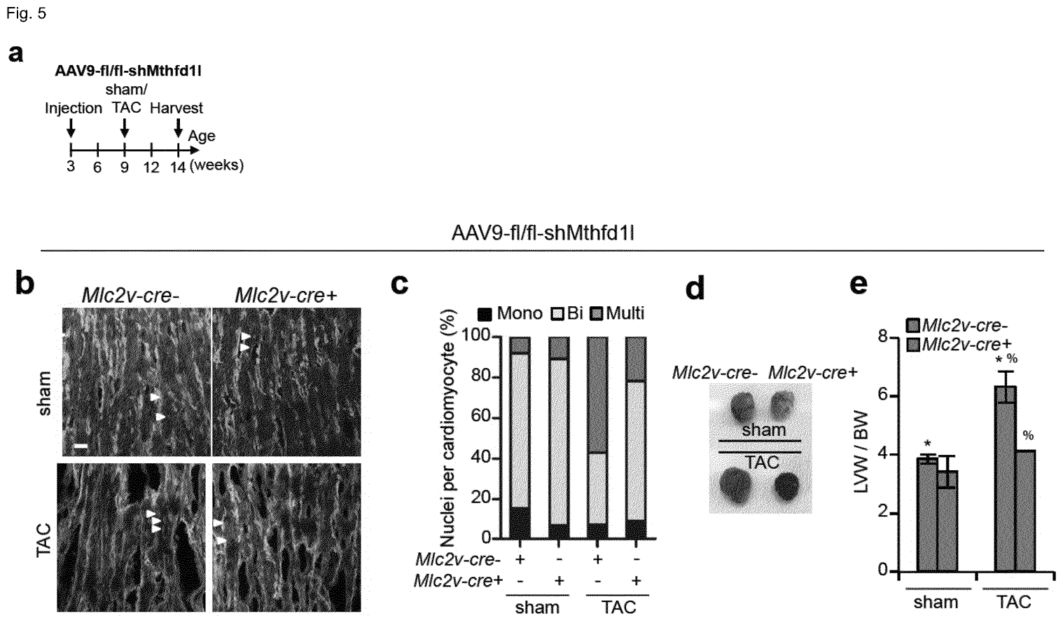

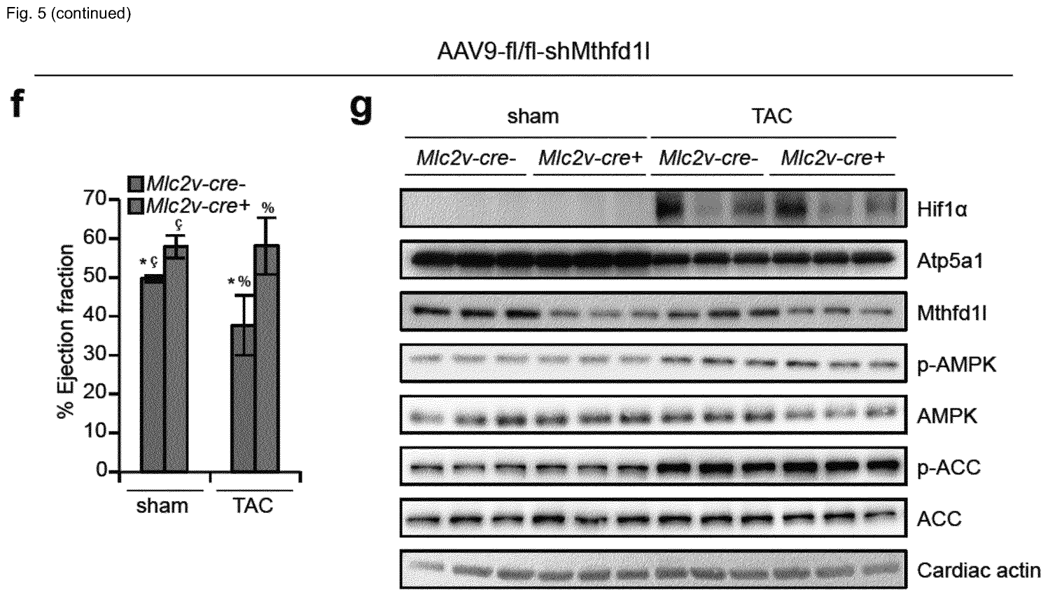

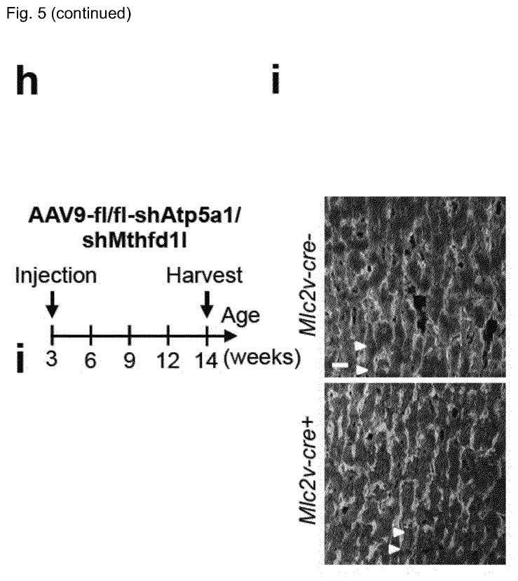

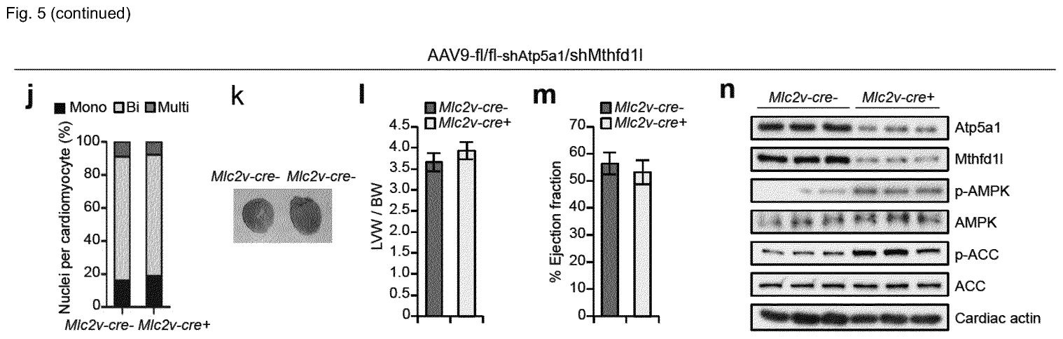

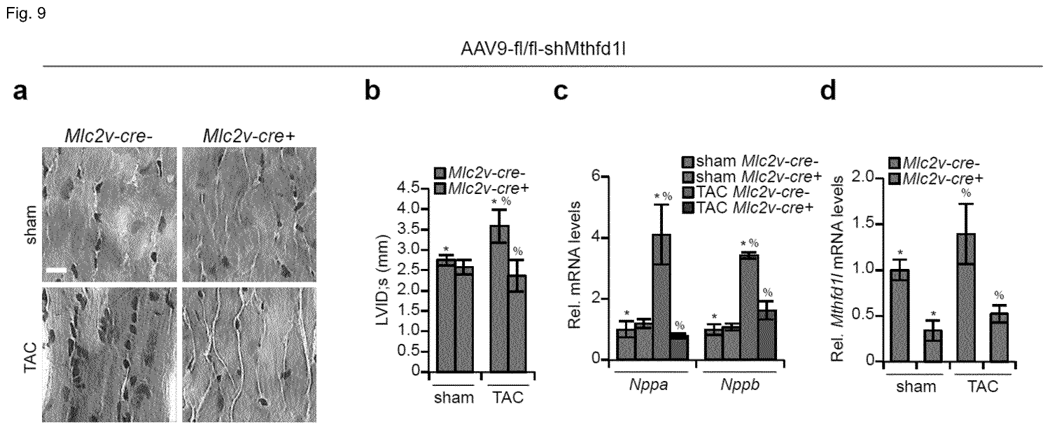

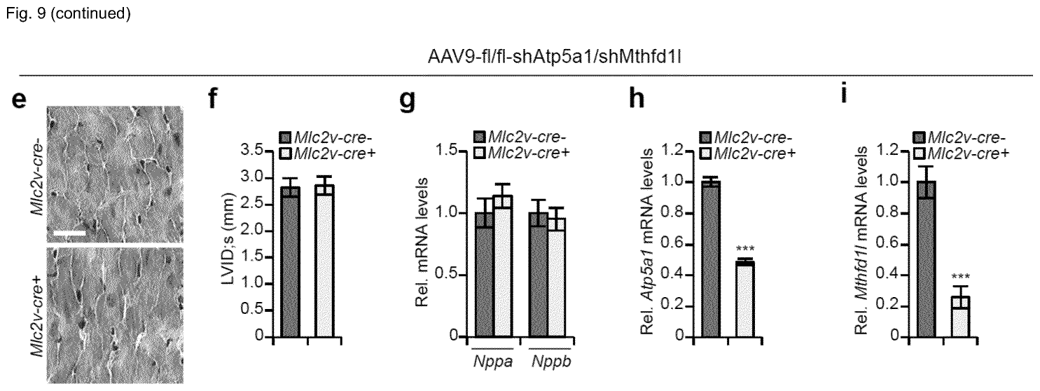

The inventors' study identifies a conserved metabolic and mechanistic pathway for endoreplication and growth control in hypertrophic heart disease. Mice with suppressed expression of MTHFD1L showed no increase in multinucleated cells (FIG. 5i, j and FIG. 9e) normal ventricular size (FIG. 5k and FIG. 9f), left ventricular weight:body weight ratio (FIG. 5l), hypertrophic marker gene expression (FIG. 9g) and systolic left ventricular function (Example 4).

A first aspect of the invention relates to an agent for use in prevention or treatment of hypertrophic heart disease, wherein the agent is selected from
a. a non-agonist biopolymer ligand specifically binding to, and capable of abrogating the biological function of, MTHFD1L protein;
b. a nucleic acid capable of specifically suppressing expression of the MTHFD1L gene.

In certain embodiments, the non-agonist biopolymer ligand is an antibody, antibody fragment, antibody-like molecule or aptamer.

Aptamers are oligonucleotide or peptide molecules that bind to a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications.

In certain embodiments, the non-agonist biopolymer ligand is a human antibody or a humanized antibody.

In certain embodiments, the binding of the non-agonist biopolymer ligand to MTHFD1L is characterized by a K_{D} of smaller than (<) 10⁻⁷, particularly K_{D}<10⁻⁸, more particularly K_{D}<10⁻⁹.

In certain embodiments, the nucleic acid capable of specifically suppressing expression of MTHFD1L is a small-interference RNA (siRNA) or an antisense oligonucleotide.

A second aspect of the invention relates to a nucleic acid molecule encoding the agent according to the first aspect for use in prevention or treatment of hypertrophic heart disease.

In certain embodiments, the nucleic acid molecule is a DNA molecule or an RNA molecule.

A third aspect of the invention relates to a nucleic acid expression vector comprising the nucleic acid molecule of the second aspect for use in prevention or treatment of hypertrophic heart disease.

In certain embodiments, the expression vector is selected from:
a. a nucleic expression construct selected from a DNA plasmid, a double stranded linear DNA, and a single stranded RNA, wherein optionally said nucleic acid expression construct is encapsulated in a lipid vesicle, and
b. a viral vector, particularly a lentiviral vector, a herpes viral vector, an adenoviral vector and an adeno-associated viral vector.

In certain embodiments, the hypertrophic heart disease is ischemic heart disease or hypertrophic heart disease associated with an elevated risk of infarction.

In certain embodiments, the hypertrophic heart disease indication is classified as restrictive cardiomyopathy, pulmonary heart disease, coronary artery disease, renal artery stenosis, aortic stenosis or aneurysm, peripheral arterial disease, hypertensive heart disease, congenital heart disease, vascular disease, valvular heart disease.

In certain embodiments, the hypertrophic heart disease is caused by or associated with hypoxia, ischemia, hypertension, stenosis, aneurysm or blockage of major blood vessels, embolisms and thrombosis, or stressors leading to cardiac pressure and/or volume overload.

A fourth aspect of the invention relates to a pharmaceutical composition for use in prevention or treatment of hypertrophic heart disease in a patient, comprising the agent according to the first aspect, the nucleic acid molecule according to the second aspect, or the nucleic acid vector according to the third aspect, and a pharmaceutically acceptable carrier. In certain embodiments, the pharmaceutical composition is formulated as an administration form for parenteral administration. In certain embodiments, the pharmaceutical composition is formulated for intravenous administration.

A fifth aspect of the invention relates to a method for identifying an MTHFD1L inhibitor, said method comprising the steps of
contacting MTHFD1L with a small molecule,
measuring the activity of MTHFD1L for said small molecule, and
selecting a small molecule as an MTHFD1L inhibitor, wherein enzymatic activity of MTHFD1L is diminished by at least 80%, particularly by at least 90%.

Similarly, a dosage form for the prevention or treatment of heart disease is provided, comprising a non-agonist ligand or antisense molecule according to any of the above aspects or embodiments of the invention.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

Topical administration is also within the scope of the advantageous uses of the invention. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1st Ed. CRC Press 1989; ISBN-13: 978-0824781835).

### Pharmaceutical Composition and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition can be formulated for oral administration, parenteral administration, or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Items

1. An agent for use in prevention or treatment of hypertrophic heart disease, wherein the agent is selected from
   a. a non-agonist biopolymer ligand specifically binding to MTHFD1L;
   b. a nucleic acid capable of specifically suppressing expression of MTHFD1L.
2. The agent for use in prevention or treatment of hypertrophic heart disease according to item 1, wherein the non-agonist biopolymer ligand is an antibody, an antibody fragment, an antibody-like molecule or an aptamer.
3. The agent according to item 1 or 2 for use in prevention or treatment of hypertrophic heart disease, wherein the non-agonist biopolymer ligand is a human antibody or a humanized antibody.
4. The agent according to any one of the preceding items for use in prevention or treatment of hypertrophic heart disease, wherein the binding of the non-agonist biopolymer ligand to MTHFD1L is characterized by a K_{D} of smaller than (<) 10⁻⁷, particularly K_{D}<10⁻⁸, more particularly K_{D}<10⁻⁹.
5. The agent for use in prevention or treatment of hypertrophic heart disease according to item 1, wherein the nucleic acid capable of specifically suppressing expression of MTHFD1L is a small-interference RNA (siRNA) or an antisense oligonucleotide.
6. A nucleic acid molecule encoding the agent according to any one of the preceding items 1 to 4 for use in prevention or treatment of hypertrophic heart disease.
7. The nucleic acid molecule for use in treatment of hypertrophic heart disease according to item 5, wherein the nucleic acid molecule is a DNA molecule or an RNA molecule.
8. A nucleic acid expression vector comprising the nucleic acid molecule of item 5 or 6 for use in prevention or treatment of hypertrophic heart disease.
9. The nucleic acid expression vector for use in prevention or treatment of hypertrophic heart disease according to item 7, wherein the expression vector is selected from:
   a. a nucleic expression construct selected from a DNA plasmid, a double stranded linear DNA, and a single stranded RNA, wherein optionally said nucleic acid expression construct is encapsulated in a lipid vesicle, and
   b. a viral vector, particularly a lentiviral vector, a herpes viral vector, an adenoviral vector and an adeno-associated viral vector.
10. The agent according to any one of items 1 to 4, the nucleic acid molecule according to item 6 or 7, or the nucleic acid vector according to item 7 or 8 for use in prevention or treatment of hypertrophic heart disease, wherein the hypertrophic heart disease is ischemic heart disease or hypertrophic heart disease associated with an elevated risk of infarction.
11. The agent according to any one of items 1 to 4, the nucleic acid molecule according to item 6 or 7, or the nucleic acid vector according to item 7 or 8 for use in prevention or treatment of hypertrophic heart disease, wherein the hypertrophic heart disease indication is classified as restrictive cardiomyopathy, pulmonary heart disease, coronary artery disease, renal artery stenosis, aortic stenosis or aneurysm, peripheral arterial disease, hypertensive heart disease, congenital heart disease, vascular disease, valvular heart disease.
12. The agent according to any one of items 1 to 4, the nucleic acid molecule according to item 6 or 7, or the nucleic acid vector according to item 7 or 8 for use in prevention or treatment of hypertrophic heart disease, wherein the hypertrophic heart disease is caused by or associated with hypoxia, ischemia, hypertension, stenosis, aneurysm or blockage of major blood vessels, embolisms and thrombosis, or stressors leading to cardiac pressure and/or volume overload.
13. A pharmaceutical composition for use in prevention or treatment of hypertrophic heart disease in a patient, comprising the agent according to any one of the preceding items 1 to 4, the nucleic acid molecule according to item 5 or 6, or the nucleic acid vector according to item 7 or 8, and a pharmaceutically acceptable carrier, particularly formulated as an administration form for parenteral administration, more particularly for intravenous administration.
14. A method for identifying an MTHFD1L inhibitor, said method comprising the steps of
   contacting MTHFD1L with a small molecule,
   measuring the activity of MTHFD1L for said small molecule, and
   selecting a small molecule as an MTHFD1L inhibitor, wherein enzymatic activity of MTHFD1L is diminished by at least 80%, particularly by at least 90%.

The invention is further illustrated by the following examples and figures, from which further embodiments and advantages can be drawn. These examples are meant to illustrate the invention but not to limit its scope.

### DESCRIPTION OF THE FIGURES

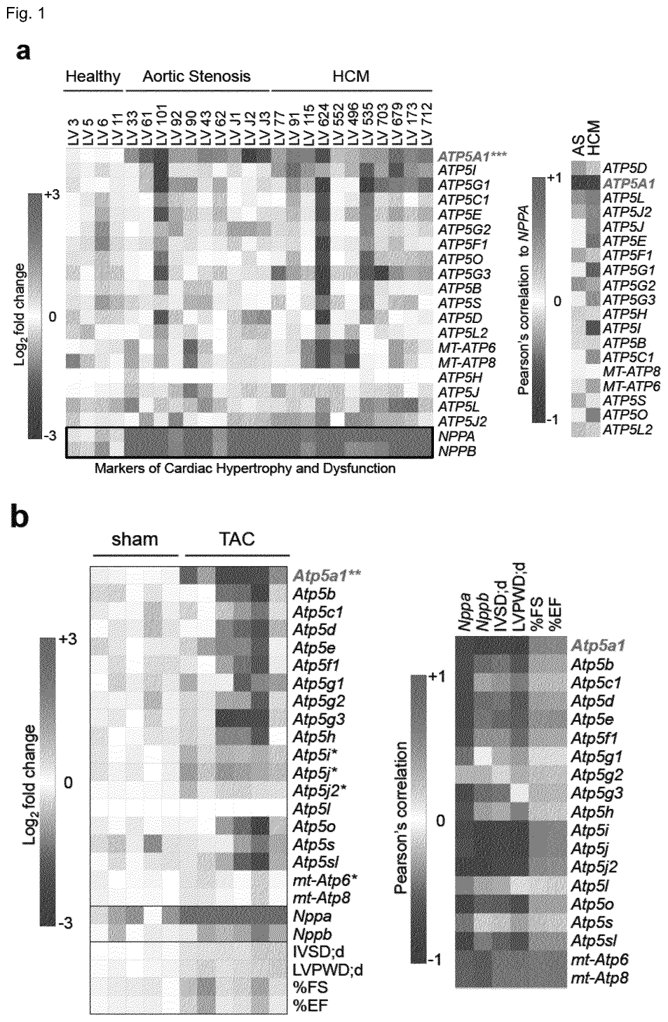

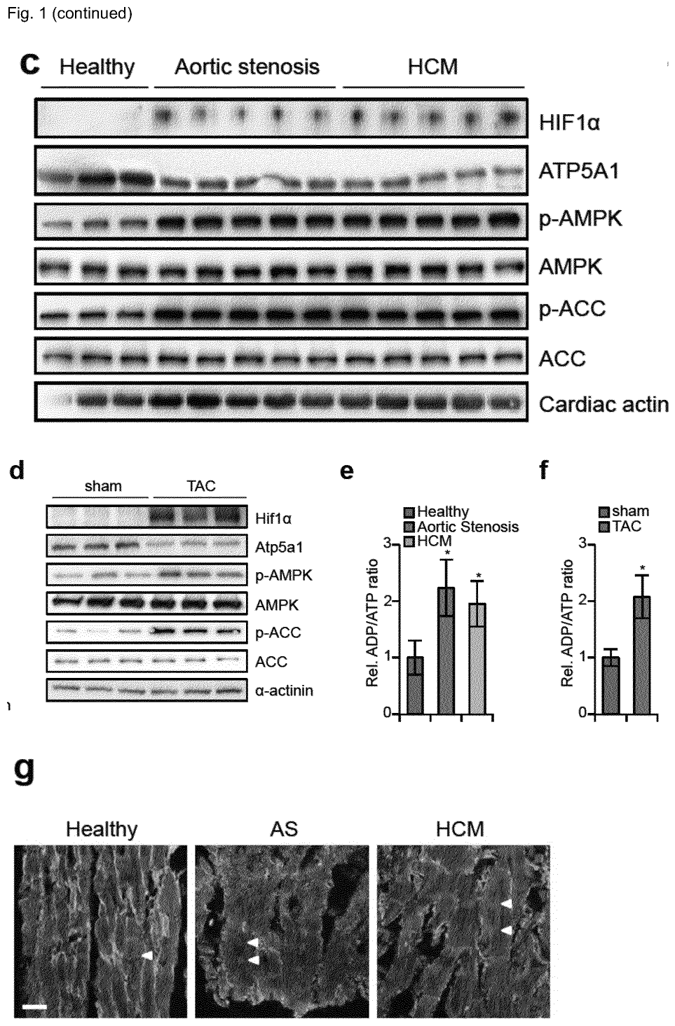

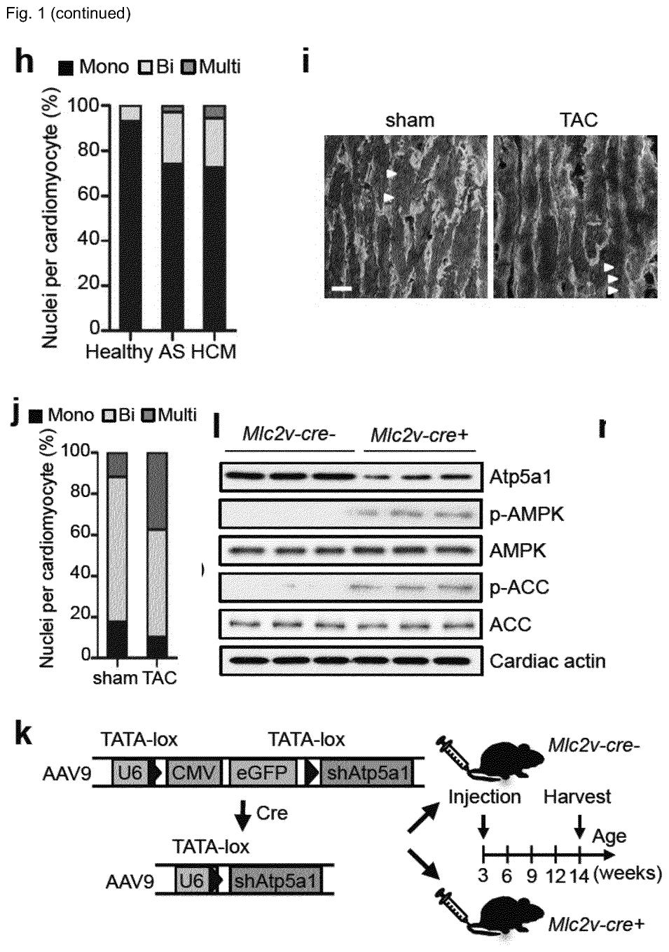

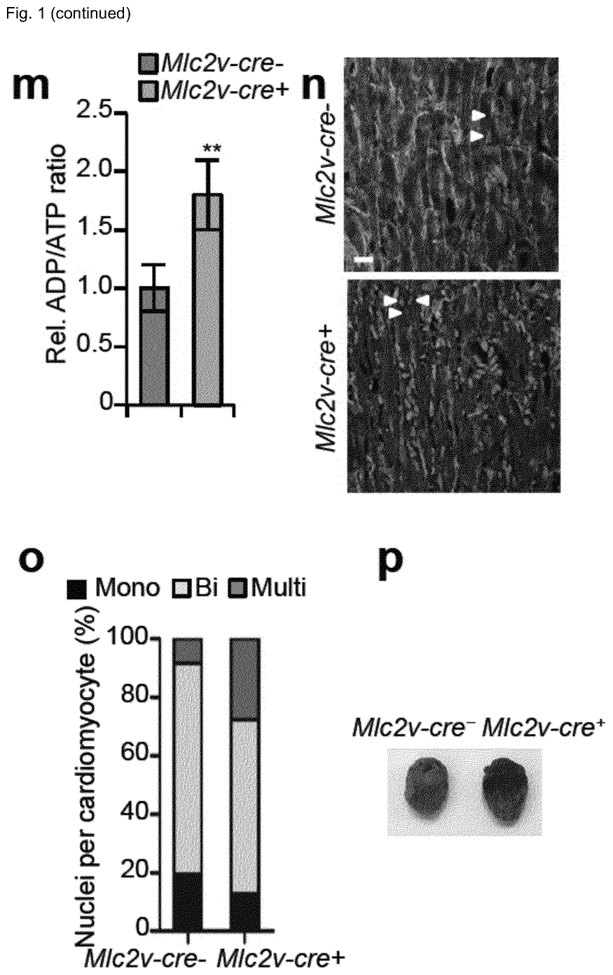

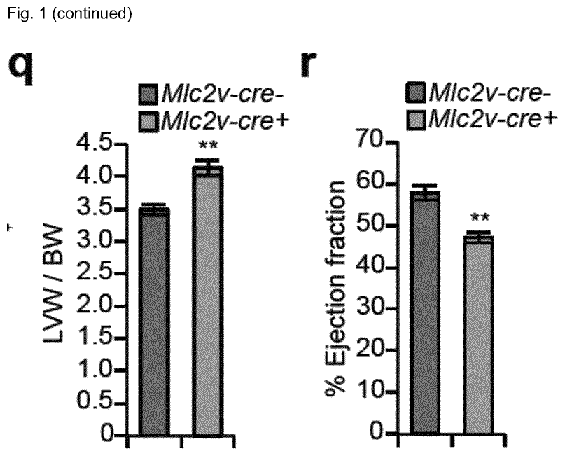

FIG. 1 F₁F₀ ATP synthase controls endoreplication and multinucleation in pathologic growth. a, Expression of genes coding for ATP synthase subunits and markers of cardiac hypertrophy and dysfunction (NPPA and NPPB) in left ventricular biopsies from hypertrophic cardiomyopathy (HCM) and aortic stenosis (AS) patients versus healthy controls (left panel). Pearson's correlation coefficient of genes presented in the left panel against expression of NPPA (right panel). (n=4 for healthy controls; n=10 for AS and n=11 for HCM; ***p<0.001; one-way ANOVA followed by Dunnett's post test). b, Expression of genes coding for ATP synthase subunits and markers of cardiac hypertrophy and dysfunction (Nppa and Nppb) in ventricular biopsies from mice subjected to transaortic constriction (TAC) versus sham-operated animals (left panel). The echocardiographically obtained measurements of the inter-ventricular septum diameter (IVSD; d) and left-ventricular posterior wall diameter in diastole (LVPWD; d), as well as the fractional shortening (% FS) and ejection fraction (% EF) of TAC or sham operated animals is shown below (left panel). Pearson's correlation coefficient of genes presented in the left panel against expression of Nppa, Nppb, IVSD; d, LVPWD; d, % FS and % EF (right panel). (n=5 for sham and n=6 for TAC; *p<0.05; **p<0.01; two-tailed unpaired t-test). c,d, Biopsies of left ventricles from patients with HCM and aortic stenosis and healthy controls (c) or sham- and TAC-operated mice (d) were assessed for denoted protein expression by immunoblotting. Loading is normalized to cardiac actin (c) or α-actinin (d). e,f, ADP/ATP ratio in ventricular biopsies from HCM and aortic stenosis patients versus healthy controls (e), and in ventricular biopsies from mice subjected to TAC versus sham-operated animals (f). (n=4 for healthy controls; n=10 for Aortic Stenosis and n=11 for HCM in (e); n=5 for sham and n=6 for TAC in (f); shown is mean±SEM; *p<0.05; one-way ANOVA followed by Dunnett's post test (d) or two-tailed unpaired t-test (f)). g, Left ventricular longitudinal sections from patients with aortic stenosis (AS) and hypertrophic cardiomyopathy (HCM) and healthy controls were stained for DAPI (blue), laminin (green) and α-actinin (red), and imaged by confocal microscopy. Representative fields are shown. Arrows indicate nucleation of cardiomyocytes. Scale bar is 20 μm. h, Heart sections stained as in (g) were assessed for the percentage of mono-, bi-, or multinucleated cardiomyocytes. (n=3 for each condition). i, Left ventricular longitudinal sections from sham- and TAC-operated mice were stained for DAPI (blue), laminin (green) and α-actinin (red), and imaged by confocal microscopy. Representative fields are shown. Arrows indicate nucleation of cardiomyocytes. Scale bar is 20 μm. j, Heart sections stained as in (g) were assessed for the ratio of mononucleated to multinucleated cardiomyocytes. (n=3 for each condition). k, Schematic representation of the AAV9-fl/fl-shAtp5a1 virus before and after Cre-mediated recombination (left panel) and of the experimental timeline (right panel). I, Immunoblot of ventricular lysates from Mlc2v-cre⁺ and Mlc2v-cre⁻ mice transduced with AAV9-fl/fl-shAtp5a1 using antibodies against denoted proteins. Loading is normalized to cardiac actin. m, ADP/ATP ratio measured in left ventricular samples of Mlc2v-cre⁺ and Mlc2v-cre⁻ mice transduced with AAV9-fl/fl-shAtp5a1 11 weeks after AAV9 injection. (n=8 for Mlc2v-cre⁻ and n=9 for Mlc2v-cre⁺ mice; results shown are the mean±SEM; ** p<0.01; two-tailed unpaired t-test) n, Left ventricular longitudinal sections from Mlc2v-cre⁺ and Mlc2v-cre⁻ mice transduced with AAV9-fl/fl-shAtp5a1 were stained for DAPI (blue), laminin (green) and α-actinin (red), and imaged by confocal microscopy. Representative fields are shown. Arrows indicate nucleation of cardiomyocytes. Scale bar is 20 μm. o, Heart sections stained as in (n) were assessed for the ratio of mono-, bi-, or multinucleated cardiomyocytes. (n=3 for each condition). p, Representative images of left ventricles of Mlc2v-cre⁺ and Mlc2v-cre⁻ mice transduced with AAV9-fl/fl-shAtp5a1 11 weeks after AAV9 injection. q,r, Left ventricular weight/body weight (LVW/BW) (q) and ejection fraction (r) of Mlc2v-cre⁺ and Mlc2v-cre⁻ mice transduced with AAV9-fl/fl-shAtp5a1 11 weeks after AAV9 injection. (n=8 for Mlc2v-cre⁻ and n=9 for Mlc2v-cre⁺ mice; results shown are the mean±SEM; **p<0.01; two-tailed unpaired t-test).

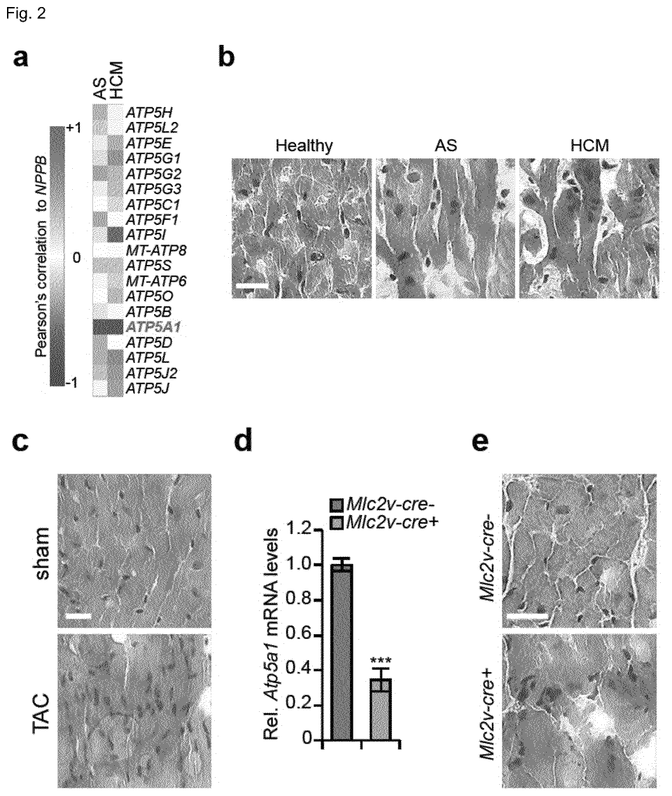

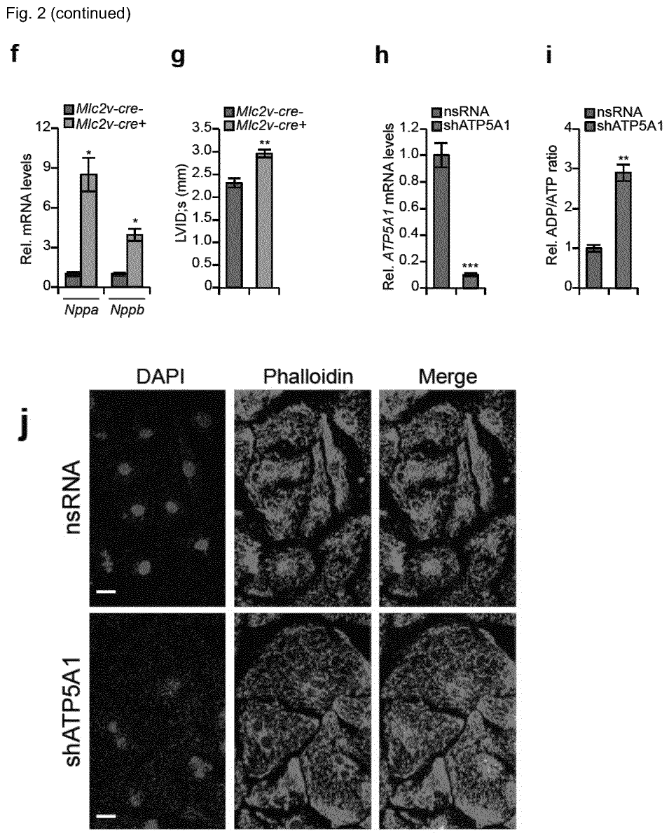

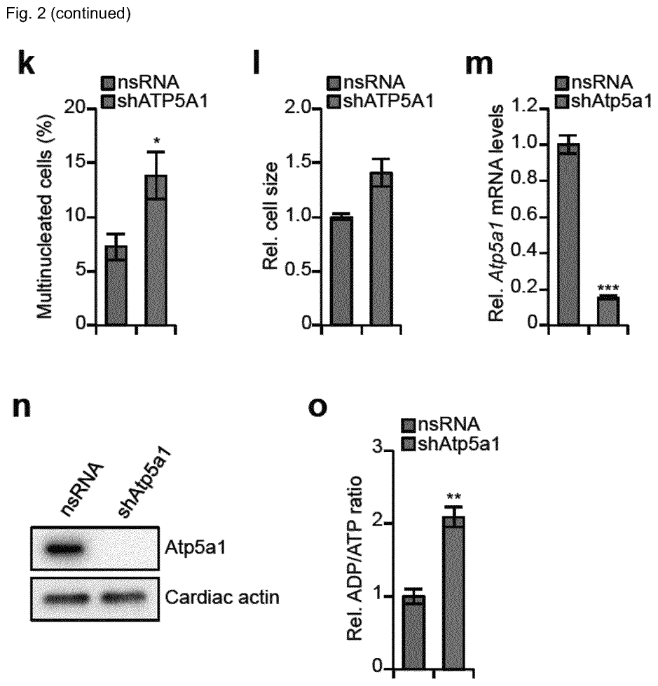

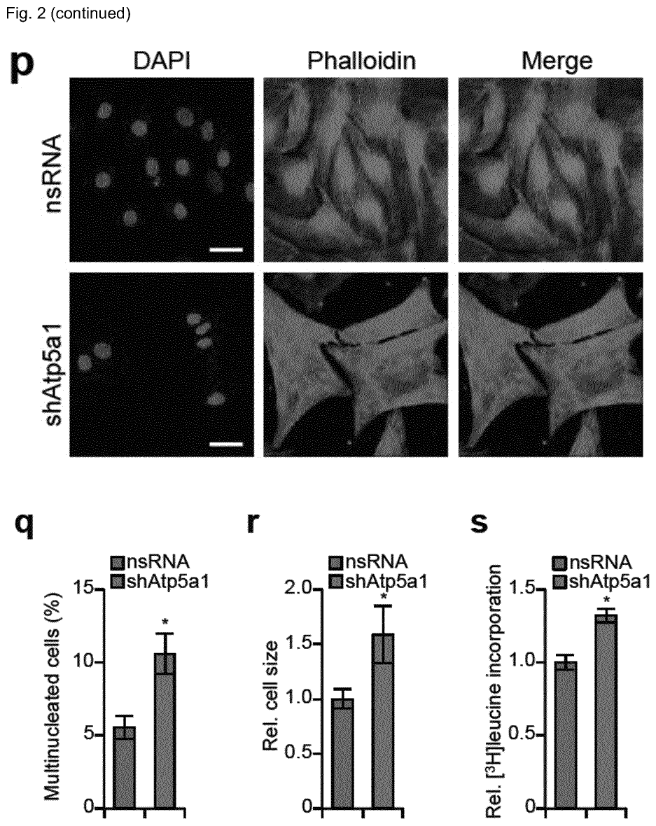

FIG. 2 Validation of ATP5A1 downregulation and cell growth. a, Pearson's correlation coefficient of ATP synthase subunit genes presented in FIG. 1a (left panel) against expression of NPPB. (n=4 for healthy controls; n=10 for AS and n=11 for HCM). b, Left ventricular sections from HCM and aortic stenosis patients and healthy controls were stained with haematoxylin and eosin (H&E) and imaged by light microscopy. Representative fields are shown. Scale bar is 200 μm. c, Left ventricular sections from sham- and TAC-operated mice were stained with H&E and imaged by light microscopy. Representative fields are shown. Scale bar is 200 μm. d, Relative expression of Atp5a1 mRNA in Mlc2v-cre⁺ and Mlc2v-cre⁻ mice transduced with AAV9-fl/fl-shAtp5a1 11 weeks after AAV9 injection. Data is normalized to Mlc2v-cre⁻ mice transduced with AAV9-fl/fl-shAtp5a1 (set as 1.0). (n=8 for Mlc2v-cre⁻ and n=9 for Mlc2v-cre⁺; shown is mean±SEM; ***p<0.001; two-tailed unpaired t-test). e, Left ventricular sections from Mlc2v-cre⁺ and Mlc2v-cre⁻ mice injected with AAV9-fl/fl-shAtp5a1 viruses were stained with H&E and imaged by light microscopy. Representative fields are shown. Scale bar is 200 μm. f, Relative expression of Nppa and Nppb mRNA in Mlc2v-cre⁺ and Mlc2v-cre⁻ mice transduced with AAV9-fl/fl-shAtp5a1 11 weeks after AAV9 injection. Data is normalized to Mlc2v-cre⁻ mice transduced with AAV9-fl/fl-shAtp5a1 (set as 1.0). (n=8 for Mlc2v-cre⁻ and n=9 for Mlc2v-cre⁺; shown is mean±SEM; *p<0.05; two-tailed unpaired t-test). g, LVID; s in Mlc2v-cre⁺ and Mlc2v-cre⁻ mice transduced with AAV9-fl/fl-shAtp5a1 11 weeks after AAV9 injection. (n=8 for Mlc2v-cre⁻ and n=9 for Mlc2v-cre⁺; shown is mean±SEM; ** p<0.01; two-tailed unpaired t-test). h, Relative expression of ATP5A1 mRNA in iPSC-derived human cardiomyocytes (iPSC-hCM) transduced with lentivirus expressing non-silencing shRNA (nsRNA) or shRNA against ATP5A1 (shATP5A1). Data is normalized to control NRCs expressing nsRNA (set as 1.0). (n=3 biological replicates per group; results shown are the mean±SD; ***p<0.001; two-tailed unpaired t-test). ADP/ATP ratio in iPSC-hCM transduced with lentivirus expressing nsRNA or shATP5A1. (n=3 biological replicates per group; shown is mean±SD; **p<0.01; two-tailed unpaired t-test). j, iPSC-hCM transduced with lentivirus expressing nsRNA or shATP5A1 were stained for DAPI and phalloidin, and imaged by confocal microscopy. Representative fields are shown. Scale bar is 20 μm. k, Quantification of percentage of multinucleated cells in iPSC-hCM treated as in (j) (n=3 biological replicates; shown is mean±SD; *p<0.05; two-tailed unpaired t-test). l, iPSC-hCM treated as in (j) were assessed for cell size using ImageJ. (n=3 independent experiments with approximately 100 cells analyzed per experiment and condition; shown is mean±SD; *p<0.05; two-tailed unpaired t-test). m, Relative expression of Atp5a1 mRNA in NRCs transduced with lentivirus expressing nsRNA or shAtp5a1. Data is normalized to control NRCs expressing nsRNA (set as 1.0). (n=3 biological replicates per group; results shown are the mean±SD; ***p<0.001; two-tailed unpaired t-test). n, NRCs transduced with nsRNA or shAtp5a1 were assessed for Atp5a1 protein levels by immunoblotting. Loading is normalized to cardiac actin. o, ADP/ATP ratio in NRCs transduced with lentivirus expressing nsRNA orAtp5a1 shRNA as indicated. (n=3 biological replicates per group; shown is mean±SD; **p<0.01; two-tailed unpaired t-test). p, NRCs transduced with lentivirus expressing nsRNA orAtp5a1 shRNA were stained for phalloidin and DAPI, and imaged by confocal microscopy. Representative fields are shown. Scale bar is 20 μm. q, Quantification of the percentage of multinucleated cells in NRCs treated as in (p) (n=3 biological replicates; shown is mean±SD; *p<0.05; two-tailed unpaired t-test). r, NRCs treated as in (p) were assessed for cell size using ImageJ. (n=3 independent experiments with approximately 100 cells analyzed per experiment and condition; shown is mean±SD; *p<0.05; two-tailed unpaired t-test). s, Evaluation of [³H]leucine incorporation in NRCs transduced with lentivirus expressing nsRNA or Atp5a1 shRNA. Data is represented as incorporated radioactivity relative to control NRCs expressing nsRNA (set as 1.0). (n=4 biological replicates per group; results shown are the mean±SEM; *p<0.05; two-tailed unpaired t-test).

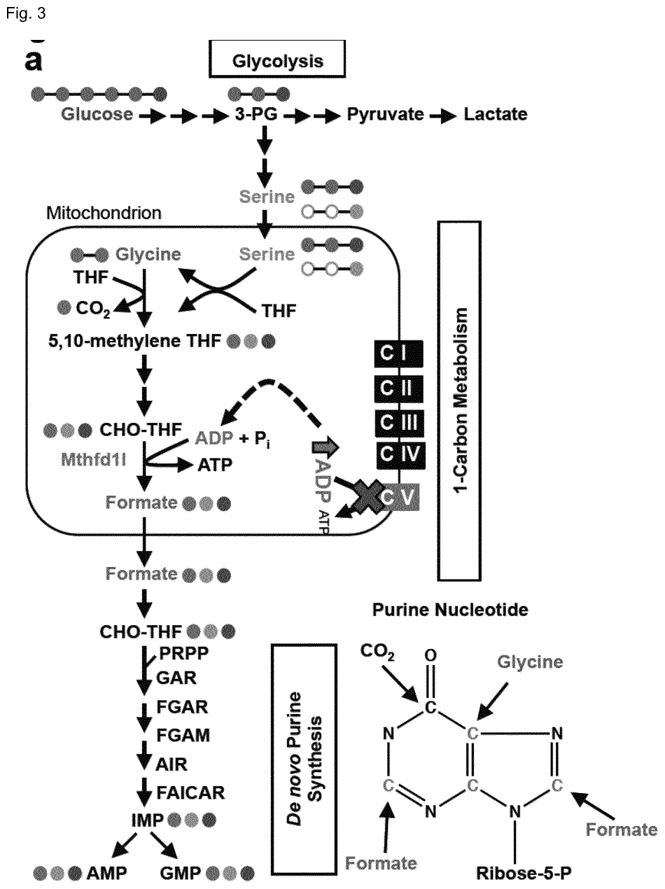

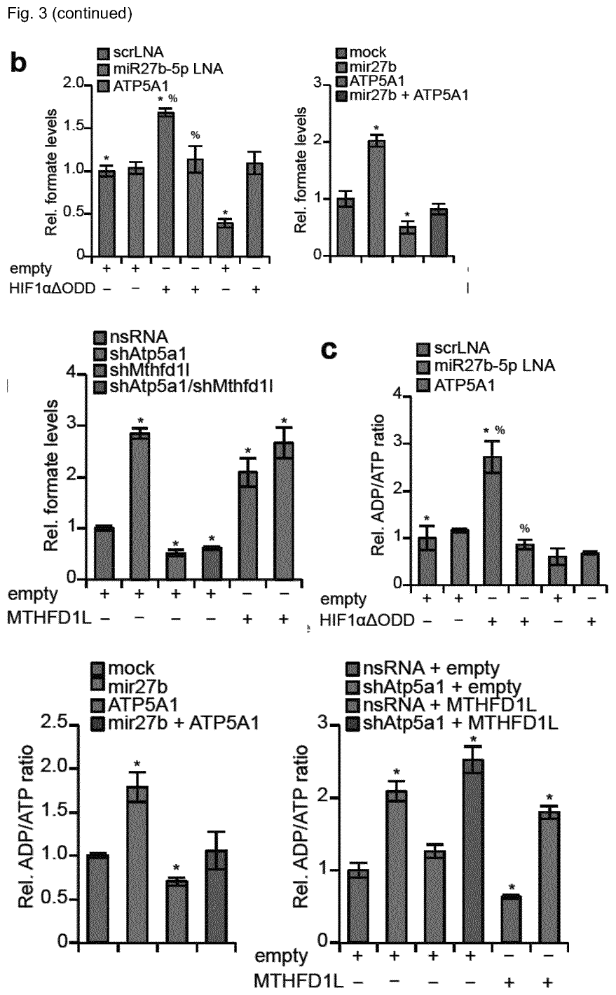

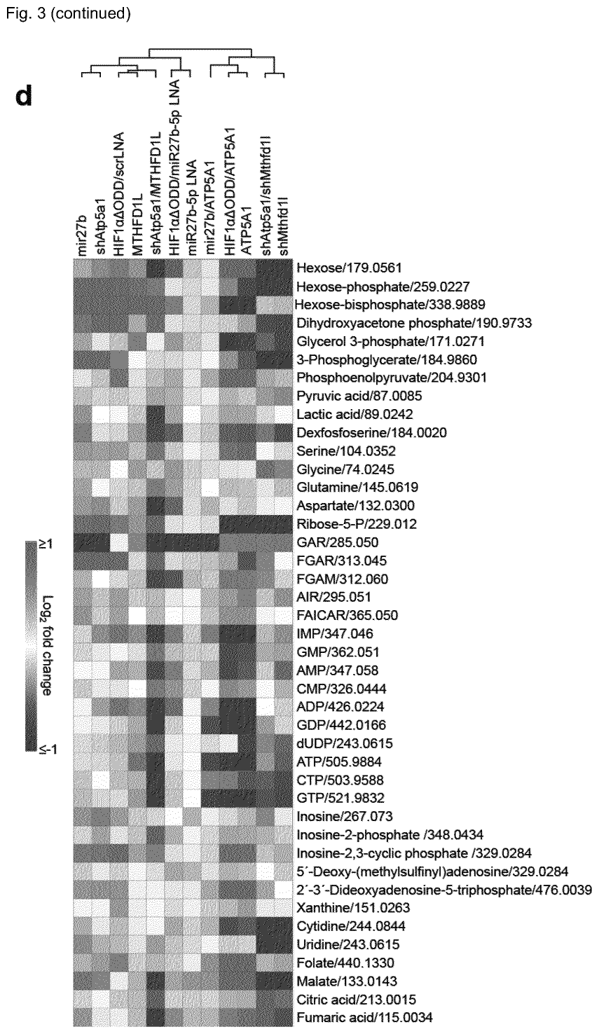

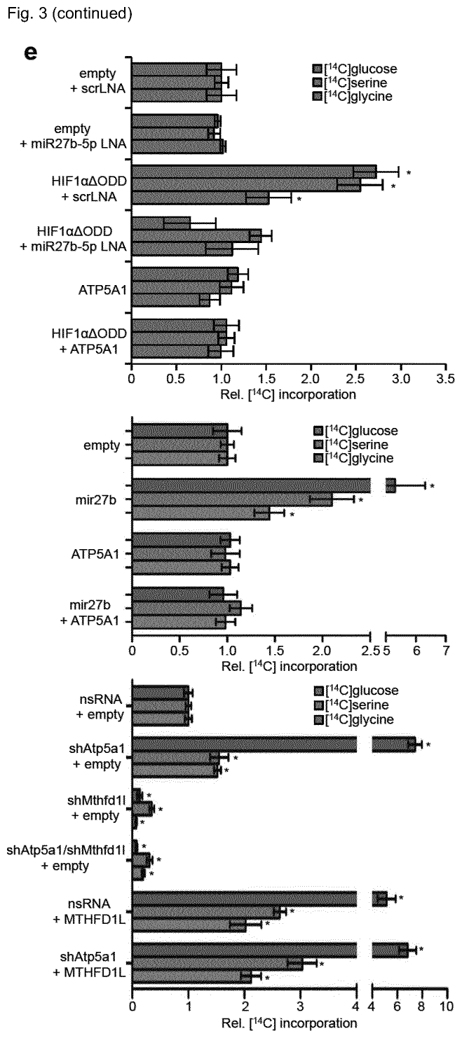

FIG. 3 ATP synthase inactivation promotes de novo nucleotide biosynthesis. a, Schematic representation of de novo purine biosynthesis pathway showing the contribution of glycolysis and 1-carbon metabolism. The origin of the carbon atoms of the purine ring is indicated. Circles represent carbon atoms and filled circles radiolabelled carbon atoms. b, Relative amount of formate in NRCs transduced and treated as denoted. (n=5 biological replicates per group; results shown are the mean±SEM; *,% p<0.05; one-way ANOVA followed by Bonferroni correction). c, Relative mitochondrial ADP/ATP ratio in NRCs transduced and treated as denoted. (n=5 biological replicates per group; results shown are the mean±SEM; *,% p<0.05; one-way ANOVA followed by Bonferroni correction). d, Heat map of relative metabolite abundance in NRCs transduced and treated as indicated. Depicted are metabolites with log₂(fold change)>0.5 compared to control treatment and adjusted p value<0.01 in at least one treatment group compared to corresponding control (n=4 biological replicates per group). e, Relative amount of [¹⁴C]carbon derived from [¹⁴C]glucose, [¹⁴C]serine and [¹⁴C]glycine incorporated into nucleic acids in NRCs transduced and treated as denoted. (n=5 biological replicates per group; results shown are the mean±SEM; *p<0.05; one-way ANOVA followed by Bonferroni correction).

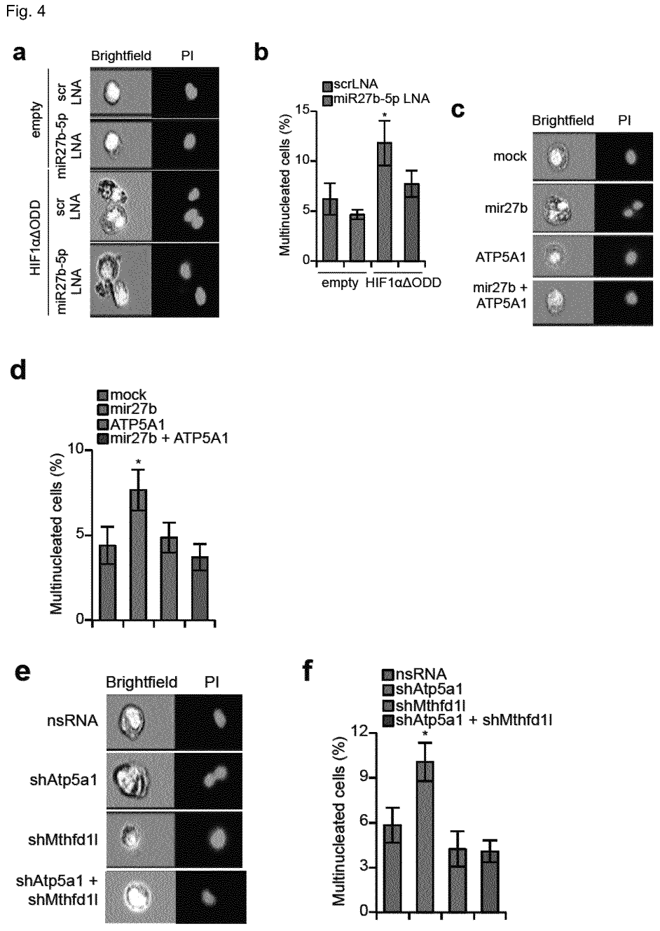

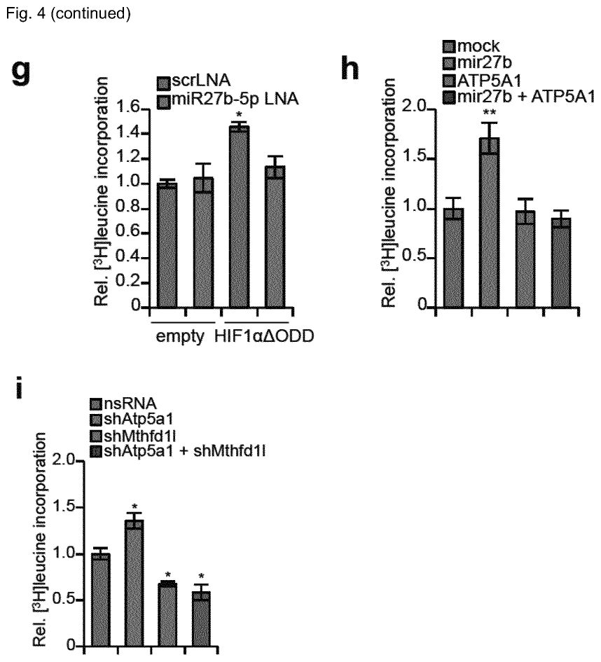

FIG. 4 ATP synthase controls cardiomyocyte karyokinesis and pathologic growth. a-f, NRCs transduced and treated as indicated were stained with propidium iodide (P1) and assessed for polyploidy by flow cytometry coupled to imaging (a,c,e) and multinucleation quantified from images (b,d,f). (n=3 biological replicates per group; results shown are the mean±SD; *p<0.05; one-way ANOVA followed by Bonferroni correction). g-i, Evaluation of [³H]leucine incorporation in NRCs transduced and treated as indicated. Data is represented as incorporated radioactivity relative to control NRCs (set as 1.0). (n=4 biological replicates per group; results shown are the mean±SEM; *p<0.05; **p<0.01; one-way ANOVA followed by Bonferroni correction (h)).

FIG. 5 MTHFD1L is necessary for multinucleation and pathologic cardiac hypertrophy and dysfunction in vivo. a, Schematic representation of the experimental timeline of Mlc2v-cre mice transduced with AAV9-fl/fl-shMthfd1l and subjected to sham or TAC surgery. b, Left ventricular longitudinal sections from sham- or TAC-operated Mlc2v-cre⁺ and Mlc2v-cre⁻ mice injected with AAV9-fl/fl-shMthfd1l viruses were stained for DAPI (blue), laminin (green) and α-actinin (red), and imaged by confocal microscopy. Representative fields are shown. Arrows indicate nucleation of cardiomyocytes. Scale bar is 20 μm. c, Heart sections stained as in (c) were assessed for the ratio of mononucleated to multinucleated cardiomyocytes. (n=3 for each condition). d, Representative images of left ventricles of sham or TAC-operated Mlc2v-cre⁺ and Mlc2v-cre⁻ mice injected with AAV9-fl/fl-shMthfd1l viruses. e,f, LVW/BW (e) and ejection fraction (f) of sham or TAC-operated Mlc2v-cre⁺ and Mlc2v-cre⁻ mice injected with AAV9-fl/fl-shMthfd1l viruses. (n=4 for sham Mlc2v-cre⁻, n=5 for sham Mlc2v-cre⁺, n=8 for TAC Mlc2v-cre⁻ and n=7 for TAC Mlc2v-cre⁺ mice; shown is mean±SEM; *, ç, % p<0.05; one-way ANOVA followed by Bonferroni correction). g, Immunoblot of ventricular lysates from sham or TAC-operated Mlc2v-cre⁺ and Mlc2v-cre⁻ mice transduced as in (b-f) using antibodies against denoted proteins. Loading is normalized to cardiac actin. h, Schematic representation of the experimental timeline of Mlc2v-cre mice co-transduced with AAV9-fl/fl-shAtp5a1 and AAV9-fl/fl-shMthfd1l. i, Left ventricular longitudinal sections from Mlc2v-cre⁺ and Mlc2v-cre⁻ mice co-transduced with AAV9-fl/fl-shAtp5a1 and AAV9-fl/fl-shMthfd1l were stained for DAPI (blue), laminin (green) and α-actinin (red), and imaged by confocal microscopy. Representative fields are shown. Arrows indicate nucleation of cardiomyocytes. Scale bar is 20 μm. j, Heart sections stained as in (j) were assessed for the ratio of mononucleated to multinucleated cardiomyocytes. (n=3 for each condition). k, Representative images of left ventricles of Mlc2v-cre⁺ and Mlc2v-cre⁻ mice co-transduced with AAV9-fl/fl-shAtp5a1 and AAV9-fl/fl-shMthfd1l. l,m, LVW/BW (l) and ejection fraction (m) of Mlc2v-cre⁺ and Mlc2v-cre⁻ mice co-transduced with AAV9-fl/fl-shAtp5a1 and AAV9-fl/fl-shMthfd1l. Data is normalized to Mlc2v-cre⁻ mice (set as 1.0). (n=6 mice per group; shown is mean±SEM). n, Immunoblot of ventricular lysates from Mlc2v-cre⁺ and Mlc2v-cre⁻ mice transduced as in i-m using antibodies against denoted proteins. Loading is normalized to cardiac actin.

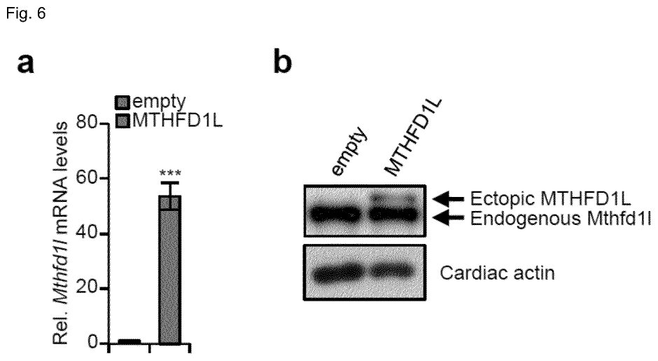

FIG. 6 Validation of ectopic MTHFD1L expression in NRCs. a, Relative expression of Mthfd1l mRNA in NRCs expressing empty control vector or ectopic MTHFD1L. Data is normalized to NRCs expressing empty control vector (set as 1.0). (n=3 biological replicates per group; results shown are the mean±SD; ***p<0.05; two-tailed unpaired t-test). b, Immunoblot of NRCs transduced with lentivirus expressing empty control vector or MTHFD1L using antibodies against Mthfd11. Loading is normalized to cardiac actin.

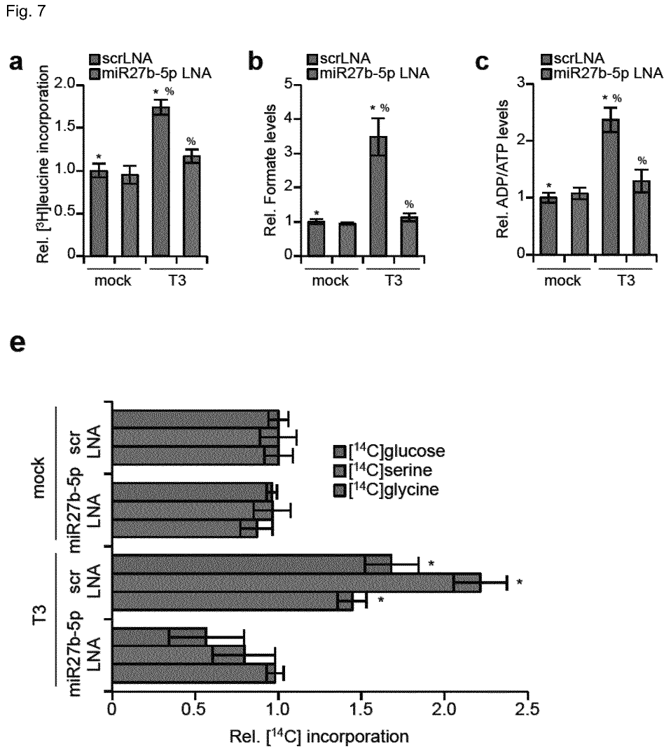

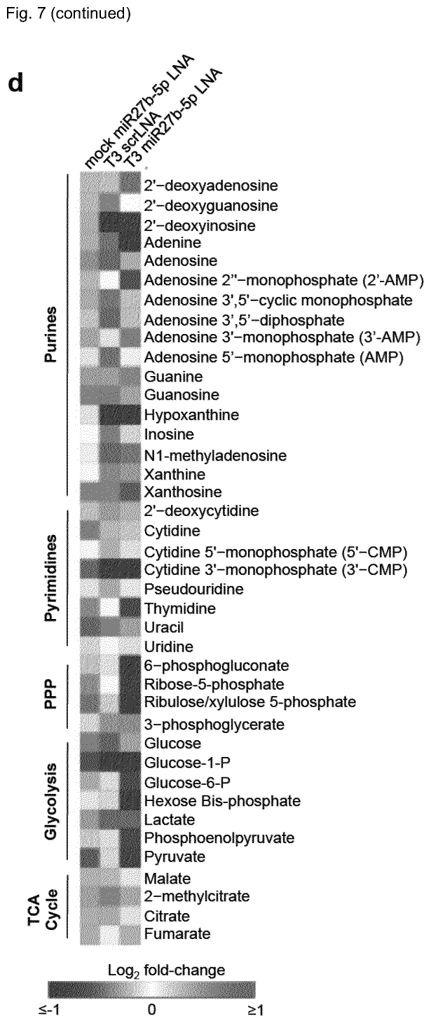

FIG. 7 T3 promotes purine biosynthesis and cell growth in a MIR27B-dependent manner. a, Evaluation of [³H]leucine incorporation in NRCs stimulated with T3 and treated with scrLNA or miR27b-5p LNAs. Data is represented as incorporated radioactivity relative to control (mock) NRCs treated with scrLNAs (set as 1.0). (n=4 biological replicates per group; results shown are the mean±SEM; *, % p<0.05; one-way ANOVA followed by Bonferroni correction). b, Relative amount of formate in NRCs stimulated with T3 and treated with scrLNA or miR27b-5p LNAs. (n=5 biological replicates per group; results shown are the mean±SEM; *,% p<0.05; one-way ANOVA followed by Bonferroni correction). c, Relative ADP/ATP ratio in NRCs stimulated with T3 and treated with scrLNA or miR27b-5p LNAs. (n=5 biological replicates per group; results shown are the mean±SEM; *,% p<0.05; one-way ANOVA followed by Bonferroni correction). d, Heat map of relative metabolite abundance in NRCs stimulated with T3 and treated with scrLNA or miR27b-5p LNAs. Depicted are metabolites with log₂(fold change)>0.4 and adjusted p value<0.01 in at least one treatment group compared to corresponding control (n=4 biological replicates per group). e, Relative amount of [¹⁴C]carbon derived from [¹⁴C]glucose, [¹⁴C]serine and [¹⁴C]glycine incorporated into nucleic acids in NRCs stimulated with T3 and treated with scrLNA or miR27b-5p LNAs. (n=5 biological replicates per group; results shown are the mean±SEM; *p<0.05; one-way ANOVA followed by Bonferroni correction).

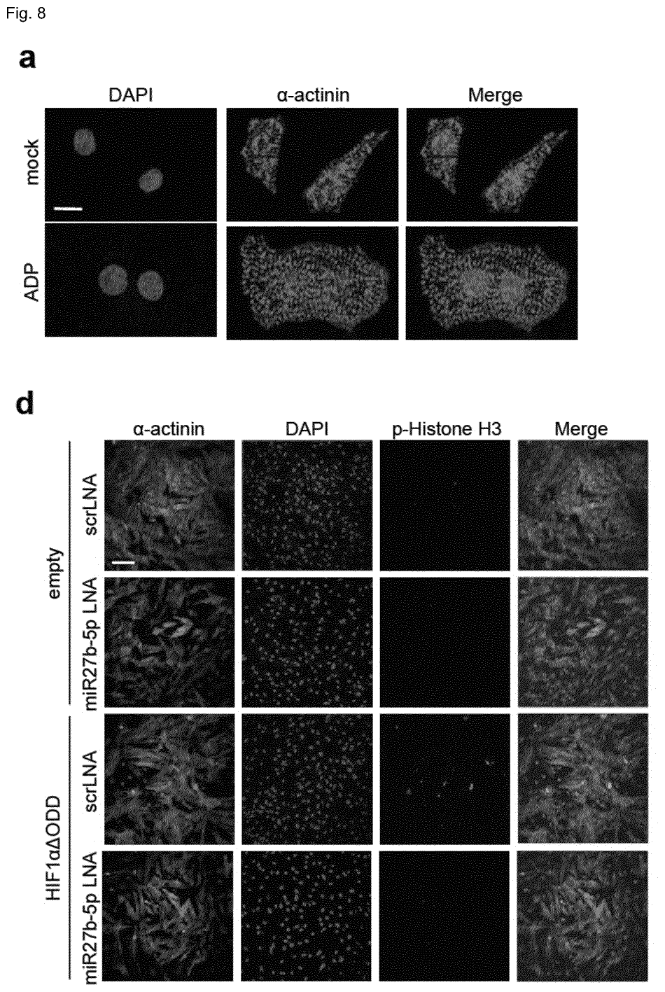

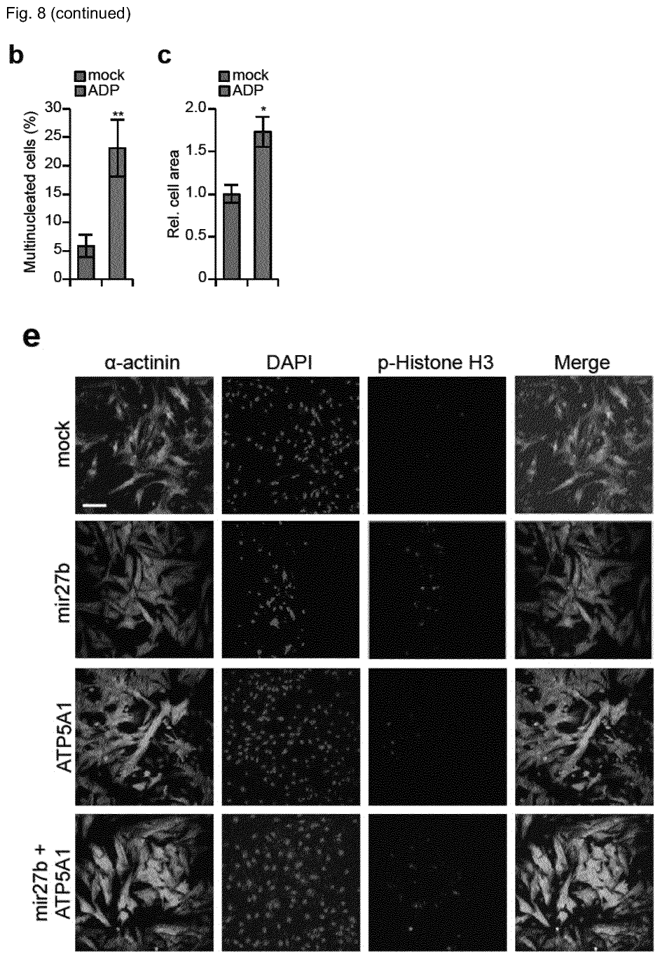

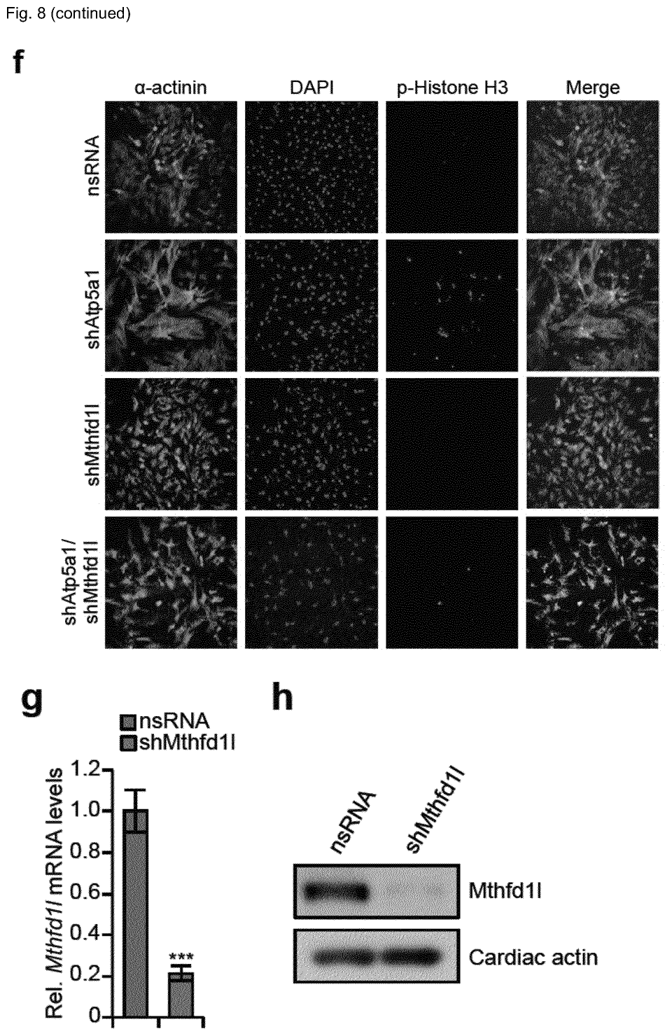

FIG. 8 HIF1α-MIR27B-ATP5A1-MTHFD1L axis promotes mitosis. a, NRCs treated with PBS or 10 μM ADP for 3 days were stained for DAPI and α-actinin and imaged by confocal microscopy. Representative fields are shown. Scale bar is 5 μm. b,c, NRCs treated and imaged as in (a) were assessed for multinucleation (b) and cell surface area using ImageJ (c). (120-150 cells were quantified per sample from n=3 independent experiments; shown is mean±SD; *p<0.05; **p<0.01; two-tailed unpaired t-test). d-f, NRCs transduced with the indicated lentiviruses were stained for the cardiac-specific marker α-actinin, DAPI and phospho-Histone H3 (p-Histone H3) to detect mitotic cells. Representative fields are shown. Scale bar is 100 μm. g, Relative expression of Mthfd1l mRNA in NRCs expressing nsRNA or shMthfd1l. Data is normalized to NRCs expressing nsRNA (set as 1.0). (n=3 biological replicates per group; results shown are the mean±SD; ***p<0.05; two-tailed unpaired t-test). h, Immunoblot of NRCs transduced with lentivirus expressing nsRNA or shMthfd1l using an antibody against Mthfd1l. Loading is normalized to cardiac actin.

FIG. 9 Validation of AAV9-fl/fl-shMthfd1l and AAV9-fl/fl-shAtp5a1 viruses and its effect on nucleation and cardiac dimension in mice. a, Left ventricular sections from sham- or TAC-operated Mlc2v-cre⁺ and Mlc2v-cre⁻ mice transduced with AAV9-fl/fl-shMthfd11 were stained with H&E and imaged by light microscopy 11 weeks post AAV9 injection. Representative fields are shown. Scale bar is 200 μm. b, Left ventricular internal dimension at systole (LVID; s) in sham- or TAC-operated Mlc2v-cre⁺ and Mlc2v-cre⁻ mice transduced with AAV9-fl/fl-shMthfd1l 11 weeks post AAV9 injection. (n=4 for sham Mlc2v-cre⁻, n=5 for sham Mlc2v-cre⁺, n=8 for TAC Mlc2v-cre⁻ and n=7 for TAC Mlc2v-cre⁺ mice; shown is mean±SEM; *, % p<0.05; one-way ANOVA followed by Bonferroni correction). c,d, Relative expression of Nppa and Nppb (c) and Mthfd1l mRNA (d) from ventricular lysates obtained from sham- or TAC-operated Mlc2v-cre⁺ and Mlc2v-cre⁻ mice transduced with AAV9-fl/fl-shMthfd1l 11 weeks post AAV9 injection. Data is normalized to sham-operated Mlc2v-cre⁻ mice (set as 1.0). (n=4 for sham Mlc2v-cre, n=5 for sham Mlc2v-cre⁺, n=8 for TAC Mlc2v-cre⁻ and n=7 for TAC Mlc2v-cre⁺ mice; shown is mean±SEM; *, % p<0.05; one-way ANOVA followed by Bonferroni correction). e, Left ventricular sections from Mlc2v-cre⁺ and Mlc2v-cre⁻ mice co-transduced with AAV9-fl/fl-shAtp5a1 and AAV9-fl/fl-shMthfd1l were stained with H&E and imaged by light microscopy 11 weeks post AAV9 injection. Representative fields are shown. Scale bar is 200 μm. f, LVID; s in Mlc2v-cre⁺ and Mlc2v-cre⁻ mice co-transduced with AAV9-fl/fl-shAtp5a1 and AAV9-fl/fl-shMthfd1l 11 weeks post AAV9 injection. (n=6 mice per group; shown is mean±SEM). g-i, Relative expression of Nppa and Nppb (g), Atp5a1 (h), and Mthfd1l mRNA (i) from ventricular lysates obtained from Mlc2v-cre⁺ and Mlc2v-cre⁻ mice co-transduced with AAV9-fl/fl-shAtp5a1 and AAV9-fl/fl-shMthfd1l 11 weeks post AAV9 injection. Data is normalized to Mlc2v-cre⁻ mice (set as 1.0). (n=6 mice per group; shown is mean±SEM; ***p<0.001; two-tailed unpaired t-test).

### EXAMPLES

### Example 1: F₁F₀ ATP Synthase Repression Drives Cardiometabolic Endoreplication and Pathologic Growth

In order to identify mediators of ATP depletion in human and mouse pathologic cardiac hypertrophy the inventors profiled all subunits of the ATP synthase complex in patient biopsies of human HCM and AS, and in mice subjected to TAC (FIG. 1a,b and FIG. 2a). Among the subunits profiled, ATP5A1 mRNA and protein was consistently repressed in both human and mouse cardiac hypertrophy and correlated significantly with upregulation of the hypertrophic markers natriuretic peptide A (NPPA), natriuretic peptide B (NPPB), and echocardiographically measured disease indicators of cardiac morphology and function (FIG. 1a-d and FIG. 2a). These changes in ATP5A1 levels correlated with activation of AMP-activated protein kinase (AMPK) through increased phosphorylation at Thr172 in the diseased myocardium of both humans and mice, resulting in phosphorylation and downstream inactivation of its direct target Acetyl-CoA-Carboxylase (ACC) (FIG. 1c,d). Critically, although historically termed as being an AMP activated kinase, AMPK is in fact more sensitive and predominantly activated by ADP. In line with repressed ATP5A1 expression and AMPK activation, ADP:ATP ratios were elevated in diseased human and mouse left ventricular biopsies (FIG. 1e,f). In addition, an increased fraction of cardiomyocytes exhibiting multinucleation, as quantified from human AS and HCM left ventricular biopsies, was observed (FIG. 1g,h and FIG. 2b). In mice, a similar increase in the fraction of multinucleated cells was observed after TAC surgery (FIG. 1i,j and FIG. 2c). The TAC protocol mimics human aortic stenosis through surgical constriction of the mouse aorta, resulting in increased blood velocity into the ventricle to impose a pressure overload stress, as encountered in aortic stenosis. To understand the physiologic implication of ATP5A1 depletion, the inventors utilized a novel adeno-associated virus (AAV)9-based system for in vivo ventricular-specific transgenesis. Briefly, delivery of an AAV containing loxP flanked genes or short-hairpin RNAs (shRNA) imparts Cre recombinase (Cre) dependency, which upon delivery to tissue-specific Cre transgenic mice results in tissue-specific ectopic gene or shRNA expression. AAV9 harboring shRNAs targeting Atp5a1 (AAV9-fl/fl-shAtp5a1) was delivered to Mlc2v-Cre transgenic mice that express Cre recombinase specifically in the ventricular myocardium (FIG. 1k). AAV9-fl/fl-shAtp5a1 delivery led to repressed Atp5a1 mRNA and protein expression (FIG. 1l and FIG. 2d), with concomitant activation of Ampk and augmented phosphorylation of its downstream target Acc (FIG. 1l), increased ADP:ATP levels (FIG. 1m), cardiomyocyte multinucleation and overgrowth combined with significantly reduced cardiac contractility (FIG. 1n-r and FIG. 2e-g). Next, the inventors confirmed these findings in induced pluripotent stem cell (iPSC)-derived human cardiomyocytes and primary neonatal rat cardiomyocytes (NRC) (FIG. 2h-s). Human and rat cardiomyocytes were stained with DAPI to visualize nuclei, and phalloidin to label filamentous actin and outline the cell surface. As noted in FIG. 2h-s, knockdown of ATP5A1 with shRNAs in human and rat heart cells resulted in increased ADP:ATP ratio and cardiomyocyte multinucleation, concomitant to cell overgrowth as quantified by 2D cell size analysis and leucine incorporation, a readout for protein synthesis as an indirect measure of growth. Thus, ATP5A1 depletion is sufficient to induce endoreplication and multinucleation to drive pathologic cardiac growth and compromise cardiac function.

### Example 2: Mitochondrial ADP Drives Purine Biosynthesis

Given that ATP synthase inactivation elevates intra-mitochondrial ADP levels, the inventors explored if ADP in the absence of substrate competition from ATP synthase could be rechanneled to MTHFD1L, an intra-mitochondrial rate-limiting enzyme of the 1-carbon pathway that accounts for 70-99% of formate produced in cells. MTHFD1L utilizes ADP as a rate-limiting cofactor for the hydrolysis of 10-formyltetrahydrofolate (CHO-THF) to formate (FIG. 3a). To test this, the inventors measured formate and mitochondrial ATP and ADP levels in genetic Hif1α, miR27b, Atp5a1 and Mthfd1l gain- and loss-of-function settings (FIG. 3b,c, FIG. 6a,b). Increased formate generation and ADP:ATP ratios correlated directly with ectopic Hif1α or miR27b expression, and shRNA-mediated Atp5a1 knockdown (FIG. 3b,c). Next, the inventors performed metabolomics in similar settings as above to investigate the metabolic link between activation of the HIF1a-MIR27b-ATP5A1 axis and elevated levels of multinucelation and cardiomyocyte growth (FIG. 3d). Strikingly ectopic Hif1a, miR27b or shAtp5a1 expression resulted in increased levels of glucose and glycolytic intermediates (Dihydroxyacetonephosphate, 3-Phosphoglycerate) pointing to increased glucose uptake and glycolysis, serine and glycine as two important carbon donors of formate (FIG. 3a), intermediates of the purine biosynthesis pathway (Ribose-5-phosphate, FGAR, FAICAR) as well as purine but also pyrimidine derivatives (Inosine, Xanthine, dUTP, GTP, Uridine), while concomitant miR27b-5p inactivation, depletion of Mthfd1l or ectopic ATP5A1 expression partially reversed these effects (FIG. 3d). Moreover, ectopic expression of MTHFD1L alone was not sufficient to induce the de novo purine biosynthesis pathway, supporting the view that mitochondrial ADP serves preferably as a substrate for ATP Synthase and that the induction of mitochondrial formate biosynthesis is highly dependent on repression of ATP5A1 (FIG. 3d). Similar results were observed with T3, resulting in highly miR27b-dependent hypertrophy (FIG. 7a), formate production (FIG. 7b) and ADP/ATP increase (FIG. 7c). LC-MS based analysis of the metabolome revealed increased production of purine and pyrimidine derivatives as a function of miR27b expression (FIG. 7d). Thus, ADP rechanneling connects energetic compromise to stress-induced biosynthesis pathways.

Purines are essential for nucleic acid synthesis, endoreplication and cell growth. Its de novo synthesis can be traced to the glucose that enters the cell and undergoes glycolysis to form 3-phosphoglycerate (3-PG), and further metabolized to generate serine and (indirectly) glycine via the serine biosynthesis pathway, which then serve as key 1-carbon donors through incorporation of its carbon into the purine ring (FIG. 3a). As the carbon atom incorporated into the purine ring essentially traces back to glucose carbons, the inventors followed the flux of carbon into the purine ring of nucleic acids through labeling of glucose, serine and glycine, respectively (FIG. 3e). As shown, increased incorporation of carbon atoms derived from glucose, serine and glycine was observed in the nucleic acid fraction of cells ectopically expressing HIF1αΔODD, miR27b or shAtp5a1 (FIG. 3e), while simultaneous ectopic MTHFD1L expression did not appreciably increase contribution of labeled carbons to the nucleic acid fraction (FIG. 3e), in line with the obtained metabolome profile (FIG. 3d). Similar effects were observed in settings of T3-induced cell growth and miR27b-5p inactivation (FIG. 7e). These data suggest that the entire upstream network impinges on the crosstalk between ATP5A1 and MTHFD1L in regulating pathology-induced de novo nucleic acid synthesis.

### Example 3: ATP Synthase Controls Cardiomyocyte Endoreplication and Pathologic Growth

Cardiomyocyte endoreplication precedes pathologic cardiac overgrowth. To understand if the axis identified indeed contributes to cardiomyocyte multinucleation and growth through ADP, the inventors stimulated primary cardiomyocyte with ADP and observed its sufficiency to induce multinucleation in response to endoreplication, as visualized in immunofluorescent staining with DAPI and skeletal a-Actinin (FIG. 8a) on which the percentage of multinucleated cells has been quantified (FIG. 8b), and cardiomyocyte hypertrophy by cardiomyocyte cell size quantification (FIG. 8c). To better define the underlying mechanism, the inventors analyzed cardiomyocyte ploidy and cell size in Hif1α, miR27b, Atp5a1 and Mthfd1l gain- and loss of function settings (FIG. 4a-i and FIG. 8d-h). As shown by imaging coupled flow cytometry and flow cytometry of propidium iodide (PI) stained DNA, ectopic HIF1α, mir27b or shAtp5a1 expression was sufficient to increase the population of multinucleated polyploid cells (FIG. 4a-f), an effect reverted upon simultaneous miR27b-5p LNA-mediated inactivation (FIG. 4a,b), ectopic ATP5A1 expression (Fig. c,d) or depletion of Mthfd1l by co-transduction of shMthfd1l expressing lentiviruses (4e,f). In order to directly visualize endoreplicated multinucleated cells, cardiomyocytes were stained for phosphorylated histone 3 at serine 10 (p-Histone H3) and imaged by confocal microscopy (FIG. 8d-f). Phosphorylated histone H3 marks condensed chromosomes that are characteristic of karyokinetic cells. Consistent with the flow cytometry data, ectopic HIF1αΔODD expression led to increased phosphorylated histone H3 stained nuclei which was rescued upon parallel miR27b-5p inhibition with LNAs (FIG. 8d). Furthermore, mir27b overexpression or Atp5a1 inhibition similarly led to increased numbers of karyokinetic cells compared to corresponding controls (FIG. 8e,f). shRNA-mediated Mthfd1l inhibition led to efficient depletion of its mRNA and protein (FIG. 8g,h) and resulted in comparable phospho-Histone H3 staining as control cells (FIG. 8f). Remarkably, simultaneous expression of ectopic ATP5A1 and mir27b, or co-transduction of NRC with shMthfd1l and shAtp5a1 lentiviruses likewise reduced the extensive pattern of phospho-Histone H3 staining as observed upon ectopic expression of mir27b or shAtp5a1 alone (FIG. 8e,f). To verify that the increase in de novo purine biosynthesis and multinucleation links directly to cell growth, the inventors performed leucine-incorporation assays under identical treatment conditions. As shown in FIG. 4g-i ectopic HIF1αΔODD or mir27b expression, or Atp5a1 knockdown led to increased leucine-incorporation, indicating increased protein synthesis and cell growth, an effect negated upon simultaneous miR27b-5p inactivation (FIG. 4g), ectopic expression of ATP5A1 (FIG. 4h). In line with the critical requirement for MTHFD1L in purine biosynthesis, the fraction of multinucleated cells was reduced upon simultaneous depletion of MTHFD1L and ATP5A1 (FIG. 4e,f) as was pathologic cell growth (FIG. 4i). Thus, the inventors' data supports the view that re-programming of the metabolic environment is sufficient to drive cardiomyocyte endoreplication, resulting in multinucleation and cell growth.

### Example 4: MTHFD1L is a Key Modulator of Pathologic Cardiac Growth

To confirm that MTHFD1L impacts cardiac pathologic hypertrophy, the inventors injected Mlc2v-cre⁻ and cre⁺ mice subjected to sham or TAC surgeries with AAV9 harboring short-hairpin RNAs targeting Mthfd1l (AAV9-fl/fl-shMthfd1l) (FIG. 5a). Consistent with the previous TAC data (FIG. 1i,j), multinucleation was significantly increased in Mlc2v-cre⁻ mice, as quantified from immunofluorescent staining (FIG. 5b,c and FIG. 9a). Concomitant with stress-induced endoreplication, Mlc2v-cre⁻ mice developed pathologic cardiac hypertrophy and systolic dysfunction after TAC surgery, evident by increased left ventricular weight:body weight ratio, increased left ventricular internal diameter in diastole (LVId) and eleveated hypertrophic marker genes and reduced ejection fraction compared to sham-operated Mlc2v-cre⁻ mice. (FIG. 5d-f and FIG. 9b,c). Strikingly, the percentage of multinucleated cells was significantly lower in TAC operated Mlc2v-cre⁺ mice compared to similarly treated Mlc2v-cre⁻ littermates (FIG. 5b,c and FIG. 9a). Accordingly, cardiac hypertrophy and systolic dysfunction was significantly reduced (FIG. 5d-f and FIG. 9b,c). qRT-PCR analysis and immunoblotting for Mthfd1l expression revealed sufficient repression of Mthf1l on both the RNA and protein level by AAV9-fl/fl-shMthfd11 transduction (FIG. 5g and FIG. 9d) coincident with resistance to TAC-induced cardiac overgrowth and contractile dysfunction.

In line with the accumulation of HIF1a in TAC treated Mlc2v-cre and Mlc2v-cre⁺ hearts, Atp5a1 expression was repressed; resulting in AMPK phosphorylation at Thr172 and hyperphosphorylation of ACC. In line with the fact that Mthfd1l repression was sufficient to prevent increased multinucleation in response to cardiometabolic endoreplication, driven by TAC mediated pressure-overload, multinucleation, overgrowth and dysfunction induced by Atp5a1 downregulation (FIG. 1g-1) was similarly prevented by the parallel inactivation of Mthfd1l in vivo (FIG. 5h-m and FIG. 9e,f). Mlc2v-cre⁺ mice co-injected with AAV9-fl/fl-shAtp5a1 and AAV9-fl/fl-shMthfd1l showed no increase in multinucleated cells (FIG. 5i, j and FIG. 9e) normal ventricular size (FIG. 5k and FIG. 9f), left ventricular weight:body weight ratio (FIG. 5l), hypertrophic marker gene expression (FIG. 9g) and systolic left ventricular function (FIG. 5m), despite efficient inhibition of Atp5A1 (FIG. 5n and FIG. 9h) when Mthfd1l was inhibited simultaneously (FIG. 9g) indicating that Mthfd1l functions downstream of Atp5a1. Thus, ATP5A1 repression and the resulting rechanneling of ADP to MTHFD1L and the consequent increase in purine biosynthesis, drives cardiometabolic endoreplication and multinucleation to enforce pathologic cardiac growth.

### DISCUSSION

In its essence, the inventors' study identifies a conserved metabolic and mechanistic pathway for endoreplication and growth control in hypertrophic heart disease. This pathway rests on competition between ATP synthase and MTHFD1L for free mitochondrial ADP, which underlies the decision to commit to pathologic cardiometabolic endoreplication and growth. The concept of substrate or cofactor shuttling forms the basis of enzyme competition wherein disparate enzymes compete for a limited common resource, which directly impacts cellular physiologic and metabolic outputs. In the context of the inventors' findings, the competition for ADP centers primarily on ATP synthase function. Given that ATP synthase is highly active in healthy cells, these cells maintain a high mitochondrial ATP:ADP ratio. In contrast, pathologies including cancer, diabetes, atherosclerosis and heart disease are singularly characterized by repressed mitochondrial respiratory chain function and metabolism resulting in a lower mitochondrial ATP:ADP ratio. As a consequence, diseased cells shift to glycolysis as an alternative means of ATP synthesis concomitant to activation of the pentose phosphate pathway, which serve in concert as critical feeder pathways for nucleotide biosynthesis. However, this reprogramming in disease serves also to provide the metabolic setting to drive the synthesis of serine and glycine, crucial 1-carbon donors for activation of the MTHFD1L-dependent 1-carbon pathway—utilizing ADP as a cofactor to catalyze the conversion of formyl-tetrahydrofolate (CHO-THF) to formate (FIG. 6m).

Mechanistically, de novo purine biosynthesis in the heart is provoked by pathologic stimuli through HIF1α activation of MIR27B-mediated repression of the alpha subunit of ATP synthase (ATP5A1) to facilitate MTHFD1L activation and consequent nucleic acid production, karyokinesis and cardiac growth (FIG. 6m). The notion that accumulating mitochondrial ADP levels activates MTHFD1L is supported by the observation that the fate of the substrate CHO-THF depends on cofactor availability. Conversion of CHO-THF to formate in mitochondria is favored over oxidation to CO2 when ADP levels are high and the reduced form of NADP+ is low whereas CO2 production is preferred when ADP levels are low.

The 1-carbon cycle not only provides carbon moieties for de novo purine biosynthesis but also for S-adenosylmethionine-dependent methylation reactions and thus might play an important role in DNA and chromatin methylation. Evidence suggests that a number of genes are differentially methylated in healthy individuals and patients with heart disease. However, as the 1-carbon cycle operates predominantly in a clock-wise feed-forward manner, the precursors of de novo purine biosynthesis lie upstream of the methylation reactions precursors, suggestive of prioritization of purine biosynthesis over the methyl cycle with respect to the 1-carbon unit incorporation. This might be of key importance in hypertrophic or highly proliferating cells as acceleration of de novo nucleotide synthesis correlates and is necessary to support these growth scenarios. Accordingly, tracer experiments with the 1-carbon donors serine and glycine as well as glucose (an upstream serine precursor (FIG. 4a) demonstrate that the de novo nucleic acid synthesis rate is elevated upon activation of HIF1α-MIR27B-ATP5A1-MTHFD1L pathway.

The criticality of de novo nucleotide synthesis in pathologic cardiac growth is further highlighted by the fact MTHFD1L inactivation prevents TAC-induced cardiac hypertrophy and dysfunction. As a direct consequence of elevated nucleic acid synthesis, an increase in the multinucleated cell populations was observed upon induction of the identified network. In line with these finding, cell cycle re-entry and progression is required for the development of cardiac pathology. Despite progression through the cell cycle and the upregulation of many proteins involved in the actomyosin ring formation for cytokinesis in the diseased state, adult cardiomyocytes are post-mitotic with minimal proliferative capacity. Instead, hypertrophic cardiomyocytes become multinucleated as they undergo karyokinesis but fail to complete cytokinesis. The inventors' link between hypertrophy and endoreplication is further supported in animal models of cardiac hypertrophy and human patients with hypertrophic hearts. Cardiomyocyte polyploidization and the resulting increase in gene copy number has been proposed to support and drive the acceleration of metabolism and biosynthetic rate to provide macromolecules necessary to support hypertrophic growth.

Taken together, the inventors' findings raise the intriguing possibility of therapeutic cardiac morphology and function renormalization through modulation of energy metabolism to restore ATP levels. This would serve to not only improve cardiac dysfunction by providing energy substrates to the contractile apparatus but would directly prevent the associated changes in cardiac morphology and growth. Given that the inventors' results suggest a role for stress-induced metabolic activation of endoreplication enforcing anabolic growth as a crucial component of the hypoxic response, these findings are likely to be of significant relevance in other pathologies such as cancer or diabetes, which are also characterized by maladaptive hypoxic responses. Thus, the activation of the HIF1α-MIR27B-ATP5A1-MTHFD1L axis represents a critical pathway underlying the molecular and metabolic basis of endomitosis and growth control in disease.

### Materials and Methods

### Animal Breeding and Maintenance

Hif1α fl/fl mice were obtained from Randall S. Johnson (University of California, San Diego, USA) and Vhl fl/fl mice were kindly provided by Rudolf Jaenisch (Massachusetts Institute of Technology, USA). The myosin light-chain (Mlc)2v-Cre (Chen et al., Development 125, 1943-1949 (1998).) line was from Ju Chen (University of California, San Diego, USA). The respective ventricular-specific mouse lines described in this manuscript were generated by crossing loxP-flanked Hif1α (Hif1α fl/fl) (Ryan, H. E., et al. Cancer Res 60, 4010-4015 (2000).) or Vhl (Vhl fl/fl) (Haase et al., Proc Natl Acad Sci USA 98, 1583-1588 (2001).) mice to myosin light-chain Mlc2v-Cre transgenic mice. The data presented in this manuscript represents studies with male mice aged from 3-20 weeks old of the C57BL/6J background. After baseline echocardiography mice were randomly assigned to groups, AAV injections, LNA delivery and echocardiography was performed blinded. In experiments including TAC surgery experiments were kept blinded over the baseline echocardiography until operation. All mice were maintained at the MRC Clinical Sciences Centre (Imperial College London), Institute of Molecular Health Sciences (ETH Zurich) and/or the Cardiovascular Assessment Facility (CAF), Department of Medicine, Department of Medicine, University of Lausanne in a specific pathogen-free facility. Maintenance and animal experimentation were in accordance with the Swiss Federal Veterinary Office (BVET) guidelines.

### Human and Mouse Ventricular Biopsies

Human heart biopsies and clinical data were generously provided by Samuel Sossalla (Georg-August-University Goettingen and DZHK, Goettingen, Germany) and Sebastian Stehr (University Hospital Leipzig, Germany). Human HCM and aortic stenosis biopsies were conducted in compliance with the local ethics committee, and written informed consent was received from all subjects prior to inclusion. Myocardial samples were obtained from patients with severe aortic stenosis undergoing aortic valve replacement and a Morrow resection from the hypertrophied left ventricular septum. Only patients without significant aortic valvular regurgitation and with preserved contractile function were included. Myocardium was also obtained from patients with severe HCM during septal myectomie surgery. The myocardial samples were acquired directly in the operating room during the surgery and immediately placed in precooled cardioplegic solution (110 mM NaCl, 16 mM KCl, 16 mM MgCl₂, 16 mM NaHCO₃, 1.2 mM CaCl₂, 11 mM glucose). Samples were frozen (−80° C.) immediately in the surgery room. Clinical data pertaining to these subjects are shown in a previous publication (Mirtschink et al., Nature 522, 444-449 (2015).).

### Transaortic Banding

9-14 week old mice were subjected to transaortic banding (TAC) through constriction of the descending aorta as described (Kassiri et al., Circ Res 97, 380-390 (2005).). The mice were monitored up to 9 weeks after surgery and their heart dimensions and functions were determined by echocardiography. In vivo miRCURY LNA Power Inhibitors were injected intraperitoneally (i.p.) into C57BL/6J mice at a dose of 10 mg/kg for 4 consecutive days at 49 days post surgery. The following In vivo miRCURY LNA Power Inhibitors were purchased from Exiqon: i-mmu-miR-27b-5p (199900). i-Cel-control_inh (199900) was used as scrambled control LNA.

### In Vivo Transthoracic Ultrasound Imaging

Transthoracic echocardiography was performed using the MS400 (18-38 MHz) probe from Vevo 2100 color doppler ultrasound machine (VisualSonics). Mice were lightly anesthetized with 1-1.5% isoflurane, maintaining heart rate at 400-550 beats per minute. The mice were placed in decubitus dorsal on a heated 37° C. platform to maintain body temperature. A topical depilatory agent is used to remove the hair and ultrasound gel is used as a coupling medium between the transducer and the skin. Hearts were imaged in the 2D mode in the parasternal long-axis view. From this view, an M-mode cursor was positioned perpendicular to the inter-ventricular septum and the posterior wall of the left ventricle at the level of the papillary muscles. Diastolic and systolic interventricular septum diameter (IVS; d and IVS; s), diastolic and systolic left ventricular posterior wall diameter (LVPW; d and LVPW; s), and left ventricular internal end-diastolic and end-systolic chamber (LVID; d and LVID; s) dimensions were measured. The measurements were taken in three separate M-mode images and averaged. Left ventricular fractional shortening (% FS) and ejection fraction (% EF) was also calculated. Fractional shortening was assessed from M-mode based on the percentage changes of left ventricular end-diastolic and end-systolic diameters. % EF is derived from the formula of (LV vol; d— LV vol; s)/LV vol; d×100. At the end of the duration of the experiment, the animals were sacrificed and the heart weight-to-body weight ratio was measured.

### Isolation and Maintenance of Primary Neonatal Rat Cardiomyocytes

Isolation of primary neonatal rat cardiomyocytes (NRC) was performed using the neonatal heart dissociation kit (130-098-373, Miltenyi Biotec) as recommended by the manufacturer. Isolated cells were pre-plated with plating medium (65% DMEM, 16% M199, 10% fetal calf serum (FCS), 5% horse serum (HS), 2% glutamine and 1% penicillin/streptomycin (P/S)) for 1.5 h to deplete of the fibroblasts. NRC were plated on Type-I Collagen-coated (Advanced Biomatrix) 3 cm dishes (Nunc) in plating medium. The plating medium was changed to maintenance medium (88% DMEM, 9% M199, 1% HS, 2% glutamine and 1% P/S) 24 h after isolation of NRCs. Cardiomyocytes were treated with 3,3′,5-Triiodo-L-thyronine (T3, T5516, Sigma Aldrich) for 6 days at a concentration of 15 nM. NRCs were stimulated with exogenous ADP (A2754, Sigma Aldrich) at a concentration of 10 μM for 3 days. NRC were randomly chosen for treatment the day after isolation.

### Human Cardiomyocyte Culture

Human induced pluripotent stem cell-derived human cardiomyocytes were purchased from Cellular Dynamics International (CMC-100-010-001) and cultured as recommended by the manufacturer. Cells were randomly chosen for transduction with lentiviruses after 6-7 days in culture and experiments were performed on day 10.

### Isolation of Mitochondria

Mitochondria were isolated using the Mitochondria Isolation Kit for Cultured Cells (Abcam). Briefly, 1.2×10⁶ NRCs were seeded per 6 cm dish and cells collected by scraping. After a freeze-thaw step to weaken the cell membrane, the cells were resuspended to a protein concentration of 5 mg/mL in Reagent A and incubated for 10 min on ice. The cells were homogenized with 30 strokes in a Dounce Homogenizer and centrifuged at 1000 g for 10 min at 4° C. The pellet was resuspended in the same volume of Reagent B than was used for Reagent A and rupturing and centrifugation was repeated. The supernatants after the two centrifugation steps were combined and centrifuged at 12000 g for 15 min at 4° C. Pellet was resuspended in 80 μL Reagent C supplemented with protease inhibitors and stored at −80° C. Mitochondrial ADP:ATP quantification was performed as recommended by the manufacturer (Abcam).

### Lentivirus Production and Infection

HEK-293T cells were transfected at 80-90% confluence with polyethylenimine (PEI) transfection reagent. 10 μg transgene, 7.5 μg pMD2.G and 6.5 μg psPAX2 were mixed with 2 ml serum-free DMEM and 45 μg PEI per 10 cm dish. After 10 min incubation at room temperature (RT) the DNA/PEI-complexes were added slowly to the cells cultured in DMEM containing 0.5% FCS and L-glutamine. Medium was changed to NRC maintenance medium 4 h after transfection. Lentiviruses were harvested 48 h after transfection and stored at −80° C. NRC were infected 20 h after isolation and incubated at 37° C./5% CO₂ overnight.

### AAV9 Constructs

For the generation of the adeno-associated viral constructs, an shRNA targeting Mthfd1l was cloned into a pSico vector where the U6 promoter-driven shRNA expression is controlled in a Cre dependent manner (Ventura et al. Proc Natl Acad Sci USA 101, 10380-10385 (2004).). The shRNAs targeting Mthfd1l were designed using the online software pSico Oligomaker (MIT, version 1.5). The following shRNA sequence was used: AAV9-fl/fl-shMthfd1l, sense: TGAATGGTGTCAGAGAATTTTTCAAGAGAAAATTCTCTGACACCATTCTTTTTTC (SEQ ID No. 001), antisense: TCGAGAAAAAAGAATGGTGTCAGAGAATTTTCTCTTGAAAAATTCTCTGACACCATTCA (SEQ ID No. 002). After in vitro validation of the knockdown efficiency of the shRNA using Adeno-Cre (Cat No ADV-005, Cell Biolabs) expression to induce Cre-mediated recombination and activation of shMthfd1l expression, the region bearing the U6 promoter, the shRNA and the TATA-lox sites of the pSico construct was amplified and ligated into NheI/XhoI linearized AAV-bGH(+) vector. All other viruses were designed and engineered by Targeted Transgenesis. AAV9 viruses were injected at a concentration of 1.2×10¹³ genome copies (GC) per kg body weight into tail veins of 3-week-old Mlc2v-cre⁻ and Mlc2v-cre⁺ mice.

In Vitro Administration of Locked Nucleic Acids (LNA) and miRNA Mimics

miRCURY LNA Power Inhibitors were added directly to the cell culture medium at a final concentration of 50 nM and fresh LNAs added every second day. The following miRCURY LNA Power Inhibitors were purchased from Exiqon: i-mmu-miR27b-5p (4101712-101). Negative Control A (199006-101) was used as non-targeting LNA.

miRIDIAN microRNA Mimics were transfected into NRCs using Lipofectamine 2000 (Invitrogen). Per well in a 96-well plate, 0.4 μL Lipofectamine 2000 was mixed with 25 μL OptiMEM (Invitrogen). After incubation of 5 min at RT, the mixture was added to an Eppendorf tube containing 6.25 nM or 12.5 nM miRNA mimics in 50 μL OptiMEM and incubated for 20 min at RT to form the complexes. 50 μL of the complexes was added to the cells cultured in 100 μL medium without antibiotics. The medium was replaced 4 h after transfection and cells were harvested 48 h after transfection. The following miRIDIAN microRNA Mimics were purchased from Dharmacon: mmu-miR27b-3p (C-310380-05-0005), mmu-miR27b-5p (C-310810-01-0005). miRIDIAN microRNA Mimic Negative Control #1 (CN-001000-01) was used as negative control.

### Transient Transfections

The pCMV6 MTHFD1L (RC223034, Origene) construct was transiently transfected into cardiomyocytes 2 days after isolation using Lipofectamine 2000 (Invitrogen) according to the manufacturer's instructions. Cells were harvested and analyzed 48 h after transfection.

### Plasmid Constructions

The pLenti-HIF1αΔODD puro lentiviral expression vector was generated by subcloning the HIF1αΔODD fragment from a pcDNA3-HA-HIF1αΔODD(401-603) plasmid (Huang et al. Proc Natl Acad Sci USA 95, 7987-7992 (1998).) into pLenti-pgk puro vector as described previously (Troilo et al. EMBO Rep 15, 77-85 (2014)). As a corresponding control the pLenti-pgk puro empty vector was used. The miR27b overexpression construct was generated by amplification of the precursor mir27b sequence and the flanking sequence of 158 bp from either end of the mir27b precursor transcript from mouse genomic DNA. The sequence was cloned into the pLKO.1 CMV puro construct provided by A. Ittner (ETH Zurich), by using EcoRI and SalI restriction sites. As a control vector the empty pLKO.1 CMV puro construct was used.

Generation of miRNA Promoter-Luciferase Constructs

A 1.0 kilobyte (kb) fragment of the mir23b, mir24-1 and mir27b promoter was amplified from mouse genomic DNA and cloned into the pGL3 luciferase reporter vector (Stratagene) between the XhoI and HindIII restriction sites. Mutation of the HRE in the mir27b promoter was generated by recombinant PCR (Elion et al. Curr Protoc Mol Biol Chapter 3, Unit 3 17 (2007).). Sense and Antisense primers were designed bearing HRE mutations, which were used to amplify the mutant 5′ and 3′ regions of the mir27b promoter, respectively. Afterwards, the 5′ and 3′ products generated from the respective PCR reactions were mixed at a 1:1 ratio and the entire fragment was amplified using primers targeting the 5′ and 3′ ends of the promoters. The wildtype constructs and the mutation of the HRE in the mir27b promoter were confirmed by DNA sequencing (Microsynth).

### Generation of Atp5a1 3′ UTR Luciferase Construct

3′ UTR of Atp5a1 was amplified from mouse cDNA and cloned into the pmirGLO Dual-Luciferase miRNA Target Expression Vector (Promega). Mutation of mir27b binding site on the 3′ UTR of Atp5a1 was generated by recombinant PCR (Casonato et al. J Lab Clin Med 144, 254-259 (2004); Krishnan, J., et al. Genes Dev 26, 259-270 (2012).). Sense and antisense primers were generated bearing mir27b binding site mutations, which were used to amplify the mutant 5′ and 3′ regions of the 3′UTR, respectively. Afterwards, the 5′ and 3′ products generated from the respective PCR reactions were mixed at a 1:1 ratio and the entire sequence was amplified using primers targeting the 5′ and 3′ ends of the Atp5a1 3′UTR. The mutation of the mir27b binding site in the 3′ UTR of Atp5a1 was confirmed by sequencing (Microsynth).

shRNA Knockdown of Mthfd1l

shRNAs targeting Mthfd1l were designed using the online software BLOCK-iT RNAi Designer (Life Technologies) and compared against the rat genome using BLAST. The shRNA was cloned into the pLKO.1 vector. Sense and antisense oligomers were resuspended to a concentration of 20 μM in Annealing Buffer (100 mM NaCl, 50 mM HEPES, pH 7.4). 5 μL of sense and antisense oligomers were mixed and annealed by incubating them in a beaker containing boiling water and letting it cool to room temperature. The annealed oligomers were ligated into the pLKO.1 vector between the AgeI and EcoRI restriction sites. 50 ng vector, 200 ng annealed oligomers, 1 μL T4 DNA ligase were mixed with 1 μL 10× ligation buffer and brought to a final volume of 10 μL with PCR-grade water. The ligation reaction was incubated overnight at 16° C. and 2 μL of the ligation reaction was transformed into competent Stbl3 bacteria by heat shock transformation. Colonies were screened for insertion of shRNA by restriction digest using NdeI and BamHI and positive clones were confirmed by DNA sequencing (Microsynth). shRNA sequences are the following:

shMthfd1I (sense 5′-3′)
(SEQ ID No. 003)
GCTTCGAGAGGCAGACATTGTCTCGAGACAATGTCTGCCTCTCGAAGC.

### Lentiviral and Expression Constructs

For knockdowns with lentiviral shRNAs the following TRC pLKO.1 shRNA vectors were used: Atp5a1 (shAtp5a1, TRCN0000076239), Hif1α (shHif1α, TRCN0000232220) and Vhl (shVhl, TRCN0000436052). pLKO.1 vector containing non-silencing shRNA (nsRNA; SHC002, Sigma) was used as a control. pLenti ATP5A1 (RC214840L1) and pCMV6 MTHFD1L (RC223034) plasmids were from Origene.

RNA-Isolation, Reverse Transcription and qRT-PCR

Samples were harvested in Trizol (Invitrogen) and total RNA isolated as recommended by the manufacturer. 750 ng RNA were reverse transcribed into cDNA using RNA to cDNA EcoDry Premix (random hexamers) kit (Clontech, Cat No 639545) following the manufacturer's instructions. Quantitative real-time PCR (qRT-PCR) reactions were set up using iTaq Universal SYBR Green Supermix (Biorad, Cat No 1725121) according to manufacturer's recommendations and run on a PikoReal Real-Time PCR machine (Thermo Scientific). Ct values were normalized against the housekeeping gene Hprt1. The following qRT-PCR primers were used:

Mouse:
Atp5a1
(SEQ ID No. 004)
5′-TCTCCATGCCTCTAACACTCG-3′
and
(SEQ ID No. 005)
5′-CCAGGTCAACAGACGTGTCAG-3′;
Atp5b
(SEQ ID No. 006)
5′-GGTTCATCCTGCCAGAGACTA-3′ 3′
and
(SEQ ID No. 007)
5′-AATCCCTCATCGAACTGGACG-3′ 3′;
Atp5c1
(SEQ ID No. 008)
5′-CCAGGAGACTGAAGTCCATCA-3′
and
(SEQ ID No. 009)
5′-AGAACCTGTCCCATACACTCG-3′;
Atp5d
(SEQ ID No. 010)
5′-TGCTTCAGGCGCGTACATAC-3′
and
(SEQ ID No. 011)
5′-CACTTGCTTGACGTTGGCA-3′;
Atp5e
(SEQ ID No. 012)
5′-CAGGCTGGACTCAGCTACATC-3′
and
(SEQ ID No. 013)
5′-GTTCGCTTTGAACTCGGTCTT-3′;
Atp5f1
(SEQ ID No. 014)
5′-AGTTCCTTTACCCTAAGACTGGT-3′
and
(SEQ ID No. 015)
5′-TTCATGCTCGACTGCTTTACTT-3′;
Atp5g1
(SEQ ID No. 016)
5′-GGTCTAATCAGGCCTGTGTCTG-3′
and
(SEQ ID No. 017)
5′-CCAGCTCCTGATCCAGCCAC-3′;
Atp5g2
(SEQ ID No. 018)
5′-CAGTGGAGTTGAAGCGACCA-3′
and
(SEQ ID No. 019)
5′-TGTCGATGTCCCTTGAAATGG-3′;
Atp5g3
(SEQ ID No. 020)
5′-TCTGCATCAGTGTTATCTCGGC-3′
and
(SEQ ID No. 021)
5′-CACCAGAACCAGCAACTCCTA-3′;
Atp5h
(SEQ ID No. 022)
5′-GCTGGGCGTAAACTTGCTCTA-3′
and
(SEQ ID No. 023)
5′-CAGACAGACTAGCCAACCTGG-3′;
Atp5i
(SEQ ID No. 024)
5′-GTTCAGGTCTCTCCACTCATCA-3′
and
(SEQ ID No. 025)
5′-CGGGGTTTTAGGTAACTGTAGC-3′;
Atp5j
(SEQ ID No. 026)
5′-TATTGGCCCAGAGTATCAGCA-3′
and
(SEQ ID No. 027)
5′-GGGGTTTGTCGATGACTTCAAAT-3′;
Atp5j2
(SEQ ID No. 028)
5′-TGCCGAGCTGGATAATGATGC-3′
and
(SEQ ID No. 029)
5′-ACCATGCTAATCCCCGAGATG-3′;
Atp5l
(SEQ ID No. 030)
5′-GAGAAGGCACCGTCGATGG-3′
and
(SEQ ID No. 031)
5′-ACACTCTGAATAGCTGTAGGGAT-3′;
Atp5o
(SEQ ID No. 032)
5′-TCTCGACAGGTTCGGAGCTT-3′
and
(SEQ ID No. 033)
5′-AGAGTACAGGGCGGTTGCATA-3′;
Atp5s
(SEQ ID No. 034)
5′-TCTGGGAATGGTTGAATACAGTG-3′
and
(SEQ ID No. 035)
5′-CTCCACACCGAAGTAGCCAC-3′;
Atp5sl
(SEQ ID No. 036)
5′-TGGCATGAGTGAGTTGGTGAC-3′
and
(SEQ ID No. 037)
5′-TGCAACAGCGTTCTTTTCTTCT-3′,
Atp6
(SEQ ID No. 038)
5′-GCCTCATTCATTACCCCAACA-3′
and
(SEQ ID No. 039)
5′-GACGGTTGTTGATTAGGCGT-3′;
Atp8
(SEQ ID No. 040)
5′-CACAAACATTCCCACTGGCA-3′
and
(SEQ ID No. 041)
5′-GTTGGGGTAATGAATGAGGCA-3′;
Ccna1
(SEQ ID No. 042)
5′-TGATGCTTGTCAAATGCTCAGC-3′
and
(SEQ ID No. 043)
5′-AGGTCCTCCTGTACTGCTCAT-3′;
Ccna2
(SEQ ID No. 044)
5′-GCCTTCACCATTCATGTGGAT-3′
and
(SEQ ID No. 045)
5′-TTGCTCCGGGTAAAGAGACAG-3′;
Ccnd1
(SEQ ID No. 046)
5′-GCGTACCCTGACACCAATCTC-3′
and
(SEQ ID No. 047)
5′-CTCCTCTTCGCACTTCTGCTC-3′;
Ccne1
(SEQ ID No. 048)
5′-GTGGCTCCGACCTTTCAGTC-3′
and
(SEQ ID No. 049)
5′-CACAGTCTTGTCAATCTTGGCA-3′;
Cdc2
(SEQ ID No. 050)
5′-AGAAGGTACTTACGGTGTGGT-3′
and
(SEQ ID No. 051)
5′-GAGAGATTTCCCGAATTGCAGT-3′;
Ckap4
(SEQ ID No. 052)
5′-TCCCGTCAGAGGGATGAGC-3′
and
(SEQ ID No. 053)
5′-GCTGGGAGTTTCTCAGGAGG-3′;
Dcun1d4
(SEQ ID No. 054)
5′-CCCTTAACAAGCTGAATCTGACA-3′
and
(SEQ ID No. 055)
5′-GGCATCACTTTGCTAAAGCAGT-3′;
Glut1
(SEQ ID No. 056)
5′-CAGTTCGGCTATAACACTGGTG-3′
and
(SEQ ID No. 057)
5′-GCCCCCGACAGAGAAGATG-3′;
Gns
(SEQ ID No. 058)
5′-CGGTGTGCGGCTATCAGAC-3′
and
(SEQ ID No. 059)
5′-CAGGGCATACCAGTAACTCCA-3′;
Hif1α
(SEQ ID No. 060)
5′-TGCTCATCAGTTGCCACTTC-3′
and
(SEQ ID No. 061)
5′-CGGCATCCAGAAGTTTTCTC-3′;
Hprt1
(SEQ ID No. 062)
5′-TCAGTCAACGGGGGACATAAA-3′
and
(SEQ ID No. 063)
5′-GGGGCTGTACTGCTTAACCAG-3′;
Ldha
(SEQ ID No. 064)
5′-TGTCTCCAGCAAAGACTACTGT-3′
and
(SEQ ID No. 065)
5′-GACTGTACTTGACAATGTTGGGA-3′;
Mthfd1l
(SEQ ID No. 066)
5′-GCGGAGAGGATGAGATCATAGA-3′
and
(SEQ ID No. 067)
5′-GTCACCCCGTCCACATCTT-3′;
Nppa
(SEQ ID No. 068)
5′-AGATGAGGTCATGCCC-3′
and
(SEQ ID No. 069)
5′-AAGCTGTTGCAGCCTA-3′,
Nppb
(SEQ ID No. 070)
5′-CCAGTCTCCAGAGCAATTCAAGAT-3′
and
(SEQ ID No. 071)
5′-GCTAATTCACAAAGGACTCGAGGT-3′;
Pdk1
(SEQ ID No. 072)
5′-GGACTTCGGGTCAGTGAATGC-3′
and
(SEQ ID No. 073)
5′-CGCAGAAACATAAACGAGGTCT-3′;
pre-mir27b
(SEQ ID No. 074)
5′-TGCAGAGCTTAGCTGATTGG-3′
and
(SEQ ID No. 075)
5′-CCTTCTCTTCAGGTGCAGAAC-3′;
St14
(SEQ ID No. 076)
5′-GCGGGACTCAAGTACAACTCC-3′
and
(SEQ ID No. 077)
5′-CATTCCGATAATGGAAGTGCCA-3′;
Vegfa
(SEQ ID No. 078)
5′-GCACATAGAGAGAATGAGCTTCC-3′
and
(SEQ ID No. 079)
5′-CTCCGCTCTGAACAAGGCT-3′;
Human:
ATP5A1
(SEQ ID No. 080)
5′-CTGTAGGCAGGAAAATAATAGG-3′
and
(SEQ ID No. 081)
5′-GGGTGAGAGAGATGGAGACC-3′;
ATP5B
(SEQ ID No. 082)
5′-CCTGTCAGGGACTATGCGG-3′
and
(SEQ ID No. 083)
5′-TCCTTACTGTGCTCTCACCCA-3′;
ATP5C1
(SEQ ID No. 084)
5′-GGTAGCGGCAGCAAAATATGC-3′
and
(SEQ ID No. 085)
5′-CCACACAGTCCTCGATCTGAG-3′;
ATP5D
(SEQ ID No. 086)
5′-TCCCACGCAGGTGTTCTTC-3′
and
(SEQ ID No. 087)
5′-GGAACCGCTGCTCACAAAGT-3′;
ATP5E
(SEQ ID No. 088)
5′-GTGGCCTACTGGAGACAGG-3′
and
(SEQ ID No. 089)
5′-GGAGTATCGGATGTAGCTGAGT-3′;
ATP5F1
(SEQ ID No. 090)
5′-AGGTCCAGGGGTATTGCAG-3′
and
(SEQ ID No. 091)
5′-TCCTCAGGGATCAGTCCATAAC-3′;
ATP5G1
(SEQ ID No. 092)
5′-CCAGGAACCCGTCTCTCAAG-3′
and
(SEQ ID No. 093)
5′-GGAAGGCGACCATCAAACAGA-3′;
ATP5G2
(SEQ ID No. 094)
5′-CCCTCCTTGGTCAAGAGCAC-3′
and
(SEQ ID No. 095)
5′-GTATCTCCGGTCGTTTCAGCA-3′;
ATP5G3
(SEQ ID No. 096)
5′-CCAGAGTTGCATACAGACCAAT-3′
and
(SEQ ID No. 097)
5′-CCCATTAAATACCGTAGAGCCCT-3′;
ATP5H
(SEQ ID No. 098)
5′-GCTGGGCGAAAACTTGCTCTA-3′
and
(SEQ ID No. 099)
5′-CCAGTCGATAGCTGGTGGATT-3′;
ATP5I
(SEQ ID No. 100)
5′-CAGGTCTCTCCGCTCATCAAG-3′
and
(SEQ ID No. 101)
5′-GCCCGAGGTTTTAGGTAATTGT-3′;
ATP5J
(SEQ ID No. 102)
5′-GGAGGACCTGTTGATGCTAGT-3′
and
(SEQ ID No. 103)
5′-TGGGGTTTTTCGATGACTTCAAA-3′;
ATP5J2
(SEQ ID No. 104)
5′-CGGAGCGTTTCAAAGAGGTT-3′
and
(SEQ ID No. 105)
5′-ATGCCAGCACCATGGTAATC-3′;
ATP5L
(SEQ ID No. 106)
5′-ATGGCCCAATTTGTCCGTAAC-3′
and
(SEQ ID No. 107)
5′-TGGCGTAGTACCAAAATGTGG-3′;
ATP5L2
(SEQ ID No. 108)
5′-CGCATTTTGGTACTACACCACG-3′
and
(SEQ ID No. 109)
5′-TGCCTGTGATCTCTCTGACATAA-3′;
ATP5O
(SEQ ID No. 110)
5′-ATTGAAGGTCGCTATGCCACA-3′
and
(SEQ ID No. 111)
5′-GCTTTTCACTTTAATGGAACGCT-3′;
ATP5S
(SEQ ID No. 112)
5′-AGCAGTTGTGTGGCGTAAAGA-3′
and
(SEQ ID No. 113)
5′-CTGATGCGATCATAATCCACCTT-3′;
HPRT1
(SEQ ID No. 114)
5′-CCTGGCGTCGTGATTAGTGAT-3′
and
(SEQ ID No. 115)
5′-AGACGTTCAGTCCTGTCCATAA-3′;
MT-ATP6
(SEQ ID No. 116)
5′-CACCTACACCCCTTATCCCC-3′
and
(SEQ ID No. 117)
5′-GCCTGCAGTAATGTTAGCGG-3′;
MT-ATP8
(SEQ ID No. 118)
5′-CACCTACCTCCCTCACCAAA-3′
and
(SEQ ID No. 119)
5′-GGCAATGAATGAAGCGAACAG-3′;
CCNA1
(SEQ ID No. 120)
5′-GAGGTCCCGATGCTTGTCAG-3′
and
(SEQ ID No. 121)
5′-GTTAGCAGCCCTAGCACTGTC-3′;
CCNA2
(SEQ ID No. 122)
5′-CGCTGGCGGTACTGAAGTC-3′
and
(SEQ ID No. 123)
5′-GAGGAACGGTGACATGCTCAT-3′;
CCND1
(SEQ ID No. 124)
5′-GCTGCGAAGTGGAAACCATC-3′
and
(SEQ ID No. 125)
5′-CCTCCTTCTGCACACATTTGAA-3′;
CCNE1
(SEQ ID No. 126)
5′-GCCAGCCTTGGGACAATAATG-3′
and
(SEQ ID No. 127)
5′-CTTGCACGTTGAGTTTGGGT-3′;
CDK1
(SEQ ID No. 128)
5′-GGATGTGCTTATGCAGGATTCC-3′
and
(SEQ ID No. 129)
5′-CATGTACTGACCAGGAGGGATAG-3′;
NPPA
(SEQ ID No. 130)
5′-CAACGCAGACCTGATGGATTT-3′
and
(SEQ ID No. 131)
5′-AGCCCCCGCTTCTTCATTC-3′;
NPPB
(SEQ ID No. 132)
5′-TGGAAACGTCCGGGTTACAG-3′
and
(SEQ ID No. 133)
5′-CTGATCCGGTCCATCTTCCT-3′.

To assess mature miRNA levels, 10 ng total RNA was transcribed into cDNA using TaqMan MicroRNA Reverse Transcription Kit (4366596, Applied Biosystems) as recommended by the manufacturer. qRT-PCR was performed using TaqMan 2× Universal PCR Master Mix (4304437, Applied Biosystems) following the manufacturers instructions and run on a DNA Engine Opticon 2 (Bio-Rad). Ct values were normalized against the housekeeping genes snoRNA (rat) or snoRNA202 (mouse). The following primers from Thermo Fisher Scientific were used: miR23b-3p (ID000400), miR23b-5p (ID243680_mat) miR24-3p (ID000402), miR24-5p (ID 000488), miR27b-3p (ID000409), miR27b-5p (ID002174), snoRNA (ID001718) and snoRNA202 (ID001232).

### Chromatin Immunoprecipitation

ChIP assays were performed with material from NRCs and the assays carried out using the ChIP-IT Kit (Active Motif) according to the manufacturer's instructions and analyzed by qRT-PCR. ChIP was performed with a ChIP-grade antibody against Hif1α (mouse, ab1, Abcam). In silico promoter analyses and alignments were performed using MatInspector and DiAlignTF (Genomatix). Primer sequences used for mir27b in the ChIP were 5′-GCATGCTGATTTGTGACTTGAG-3′ (SEQ ID No. 134) and 5′-CCTCTGTTCTCCAAACTGCAG-3′ (SEQ ID No. 135).

### MicroRNA Microarray

The microRNA microarrays were performed on 3 biological replicates of Vhl cKO mice and three control mice (Vhl fl/fl), and on 3 biological replicates of Hif1α cKO mice subjected to TAC and three controls subjected to TAC surgery (TAC Hif1α fl/fl), respectively. Cardiac dimensions and function were confirmed in all mice by echocardiography. Total RNA was isolated from left ventricle and miRNAs were labelled using the miRCURY LNA microRNA Power Labelling Kit (Exiqon) and hybridized on miRNA arrays (miRXplore) that carry 1194 DNA oligonucleotides with the reverse-complementary sequence of the mature miRNAs. These arrays cover 728 human, 584 mouse, 426 rat and 122 viral miRNAs, each spotted on the arrays in quadruplicate. The Cy5-labelled miRNAs were normalized to a reference pool of miRNAs that were simultaneously labeled with Cy3. All the data are represented as ratios of logarithmic values between the diseased and control animals and deposited under GSE62418.

### ATP Quantification In Vivo

ATP was separated and quantified on an anion exchange column (Nucleosil 4000-7 PEI, 50/4 from Macherey-Nagel) with a linear gradient (0-1.5 M NaCl in 10 mM Tris-HCl, pH 8.0) using an HPLC system equipped with two independent UV-visible spectrometers (Shimadzu,). Elution of samples was monitored at 259 and 220 nm. The 220 nm wavelength was used to detect possible traces of contaminants.

### ADP/ATP Assay

4×10⁵ NRCs were seeded per well in a 3 cm dish and the assay was performed using the EnzyLight ADP/ATP Ratio Assay Kit (ELDT-100, Bioassay Systems) as recommended by the manufacturer. Luminescence was measured on a FLUOstar Omega plate reader (BMG). For ADP/ATP quantification in human and mouse heart tissue, the assay was performed using the ADP/ATP Ratio Assay Kit (ab65313, Abcam) as recommended by the manufacturer with small modifications. Briefly, the heart tissue was frozen and stored in liquid nitrogen immediately after the harvest. The tissue was powdered with a mortar and suspended in lysis buffer (10 μl/mg of tissue powder) for 5 min at room temperature. After the centrifugation at 10000 g for 1 min, the supernatant was used for the assay. Data was normalized to protein amount in the supernatant by the Bradford assay.

### ATP Synthase Enzyme Activity Assay

4×10⁵ cells were seeded per 3 cm dish. Cells were harvested by trypsinization, followed by centrifugation at 1.2 krpm for 5 min. Pellet was washed 1× with PBS (Invitrogen) and centrifuged at 1.2 krpm for 5 min. Pellet was resuspended in 100 μl PBS and frozen at −80° C. and sample preparation was continued as recommended by the manufacturer. Absorbance was measured at 340 nm and 30° C. for 3 h at 1 min intervals using a FLUOstar Omega plate reader (BMG Labtech). ATP synthase activity was normalized to protein concentration.

### Luciferase Assay

4×10⁴ NRCs were plated in a white 96-well plate and cultured for 3 days. 40 ng of wildtype or mutant pmirGLO Atp5a1 3′UTR construct was co-transfected with 6.25, 12.5 or 25 nM control or miR27b mimics using Lipofectamine 2000 (Invitrogen) as recommended by the manufacturer. Luciferase activity was measured 24 h after transfection using the Dual Luciferase Reporter Assay System (Promega) as recommended by the manufacturer on a FLUOstar Omega Microplate Reader (BMG Labtech).

To assess promoter activity, 20 ng pGL3 vector was co-transfected with 1.25 ng Renilla and 0-260 ng HIF1αΔODD. Luciferase activity was measured 36 h after transfection using the Dual Luciferase Reporter Assay System (Promega) as recommended by the manufacturer. E2F transactivation activities were measured using the Cignal Reporter Assay Kit (CCS-003L, Qiagen) according to the manufacturer's instructions using the Dual Luciferase Reporter Assay System (Promega).

### Immunoblotting

Heart tissue was solubilized in blue wonder sample buffer (3.7 M urea, 134.6 mM Tris pH 6.8, 5.4% (v/v) sodium dodecyl sulphate (SDS), 2.3% (v/v) NP-40, 4.45% (v/v) β-mercapto-ethanol, 4% (v/v) glycerol, 60 mg/L bromphenol blue) and proteins denatured for 5 min at 95° C. after homogenization using an Ultra-Turrax T10 tissue homogeniser (IKA). NRCs were washed twice with ice-cold PBS and harvested in blue wonder sample buffer. Samples were sonicated to reduce viscosity using a Bransonic 5510 Ultrasonic water bath (Branson) and boiled for 5 min. After brief centrifugation, protein lysates were loaded into 8% or 10% polyacrylamide minigels (Biorad) and transferred to nitrocellulose membrane (GE Healthcare) by wet transfer. Membranes were blocked in 5% (w/v) milk powder (Biorad) in TBST before incubation with primary antibody diluted in 5% (w/v) bovine serum albumin (BSA) in TBST for 2 hours at room temperature or overnight at 4° C. Following three washes in 5% (w/v) milk powder in TBST, membranes were incubated for 1-2 hours with the appropriate HRP conjugated secondary antibody (anti-Goat IgG HRP, 61-1620; anti-Mouse IgG HRP, 62-6520; anti-Rabbit IgG HRP, 65-6120, Invitrogen) at a dilution of 1:5000. Membranes were then washed three times with TBST before detection with ECL (Amersham) on X-ray RX NIF films (Fisher Scientific) to detect the chemiluminescence. Signal intensities were quantified by densitometry using Image J (version 1.47) (Schneider et al. Nat Methods 9, 671-675 (2012).). The following antibodies were used for immunoblotting: a-actinin (A7811, Sigma), ACC (3676, Cell Signaling), Phospho-ACC Ser79 (11818, Cell Signaling), AMPKα (5832, Cell Signaling), Phospho-AMPKα Thr172 (2535, Cell Signaling), Atp5a1 (ab14748, Abcam), cardiac actin (61075, Progen Biotechnik), Hif1α (NB100-479, Novus Biologicals), Mthfd1l (ab116615, Abcam), Rb (9309, Cell Signaling), Phospho-Rb Ser807/811 (8516, Cell Signaling) and Vhl (2738, Cell Signaling).

### Immunofluorescent Stainings

Immunofluorescent stainings were performed as described previously (Krishnan et al. Cell Metab 9, 512-524 (2009)). After fixation of NRCs with 4% paraformaldehyde (PFA)/PBS, the cells were permeabilized and incubated with primary antibodies against sarcomeric α-actinin (A7811, Sigma Aldrich) and phospho-Histone H3 (Ser10) (05-817, Cell Signaling) diluted in 2% (v/v) HS for 1 h at RT. After 3 washes with PBS for 5 min, cells were incubated with 4′,6-diamidino-2-phenylindole (DAPI; D1306, Thermo Fisher Scientific, 0.1 μg/ml), Phalloidin 555 (A34055, Molecular Probes), AlexaFluor 647 anti-mouse (A-11001, Thermo Fisher Scientific) and AlexaFluor 488 anti-mouse (A-21235, Thermo Fisher Scientific) secondary antibody for 1 h at room temperature. Dishes were mounted onto glass slides (Fisher Scientific) with a drop of ProLong Antifade (Thermo Fisher Scientific). The dishes were fixed with clear nail polish and left to dry. Fluorescent images were acquired with the SP5 confocal microscopy (Leica) using a 20× magnification. Cell size was quantified blinded using the software Image J (version 1.47).

### Immunohistochemistry

Hearts were embedded in optimal cutting temperature (OCT) compound and sectioned at 10 μm.

Sections were fixed for 10 min with 4% PFA/PBS and after 2 washes with PBS for 2 min blocked for 1 h with 2% HS/PBS for 1 h at room temperature. After permeabilisation for 10 min with 0.2%

Triton X-100/PBS, the sections were washed 3 times with PBS for 5 min and incubated with primary antibodies against sarcomeric α-actinin (A7811, Sigma Aldrich, 1:800) and Laminin (ab11575, Abcam, 1:300) diluted in 2% (v/v) HS overnight in a humidified chamber at 4° C. After 3 washes with PBS for 10 min, sections were incubated with 4′,6-diamidino-2-phenylindole (DAPI;

D1306, Thermo Fisher Scientific, 0.1 μg/ml), AlexaFluor 555 anti-mouse (A-21422, Thermo Fisher Scientific, 1:500) and AlexaFluor 488 anti-rabbit (A-11034, Thermo Fisher Scientific, 1:500) secondary antibody for 2 h at room temperature. Sections mounted with a drop of ProLong Antifade (Thermo Fisher Scientific) and fixed with clear nail polish and left to dry. Fluorescent images were acquired with the SP8 confocal microscopy (Leica) using a 20× magnification. To determine the number of nuclei per myocyte we included only myocytes that had been cut along their longitudinal axis. Nuclei that were surrounded by the extracellular matrix stain laminin were excluded from the analysis. Quantification of nuclei was performed blinded.

Cryosections were stained with hematoxylin and eosin (H&E). Slides were visualized using a Motic AE2000 with a Moticam 3.

### [³H]Leucine Incorporation Assay

[³H]leucine incorporation assay was used to measure de novo protein synthesis as an indirect readout for cell growth (Fukuzawa et al. Hypertension 35, 1191-1196 (2000).). 4×10⁵ cells were seeded per 3 cm dish. After 3 days, cells were serum-starved overnight. Next day, cells were cultured in leucine-free medium for 4h, followed by culturing in maintenance medium containing labeled L-[4,5-³H(N)]isoleucine (specific activity 30-60 Ci/mmol, ART0233, American Radiolabelled Chemicals) at a concentration of 0.5 μCi/mL for 20 h. The next day, cells were washed with PBS, trypsinized and the radioactivity was measured for 5 min in in the liquid scintillation analyzer Tri-Carb 2800TR (Perkin Elmer). Cells were counted blinded with a Neubauer chamber after trypsinization and scintillation counts were normalized to absolute cell number.

### Nucleic Acid Incorporation Assays

4×10⁵ cells were seeded per 3 cm dish. After 4 days, cells were starved of glucose for 1.5 h and serine or glycine for 3 h. For radiolabelled glucose incorporation, cells were incubated for 4 h in glucose-free DMEM (Invitrogen) containing 1 μCi/mL uniformly labeled [U-¹⁴C]glucose (specific activity 250-360 mCi/mmol, NEC042V250UC, Perkin Elmer). For radiolabelled serine incorporation, cardiomyocytes were incubated overnight in MEM (Invitrogen) supplemented with MEM Vitamin Solution (Invitrogen) and containing 0.6 μCi/mL radiolabelled serine at carbon 3 ([3-¹⁴C]serine, specific activity 50-62 mCi/mmol, NEC827050UC, Perkin Elmer). For radiolabelled glycine incorporation, cells were incubated in BME (Invitrogen) containing 1 μCi/mL uniformly radiolabelled glycine ([¹⁴C(U)]glycine, specific activity >100 mCi/mmol, NEC276E250UC, Perkin Elmer) for 4 h. After washing the cells three times with PBS, cells were harvested in Trizol (Invitrogen) and nucleic acids isolated as recommended by the manufacturer. Isolated nucleic acids were transferred to a scintillation vial containing 4 mL of the liquid scintillation cocktail Ultima Gold (Perkin Elmer) and radioactivity was measured for 5 min in the Liquid Scintillation Analyzer Tri-Carb 2800TR (Perkin Elmer). Scintillation counts were normalized to absolute nucleic acid quantity.

### Flow Cytometry

1×10⁶ NRCs were plated on a 6 cm dish (Nunc) and after 4-6 days cells were harvested by trypsinization. Cells were washed 1× with PBS and centrifuged at 80 g for 5 min. Cells were resuspended in 500 μL PBS and 5 mL cold 70% (v/v) ethanol (kept at −20° C.) was added immediately. The fixed cells were kept at 4° C. for up to 1 week. Prior to flow cytometry analysis, cells were centrifuged and washed with PBS. After centrifugation at 80 g for 5 min the cells were resuspended in 500 μL propidium iodide solution (69 μM propidium iodide in 38 mM sodium citrate, pH 7.4) containing 40 μg/mL RNase and incubated for 1 h at 37° C. Samples were run on the ImageStream (Amnis), a flow cytometer coupled to fluorescence image acquisition to obtain representative images of the flow cytometry data. The multinucleation was quantified from the ImageStream images.

### Formate Assay

4×10⁵ cells were seeded per 3 cm dish and the assay was performed using the Formate Assay Kit (Sigma). Cells were washed twice with cold PBS and collected in 25 μL Formate Assay Buffer by scraping. After centrifugation at 15000 g at 4° C. for 5 min, 20 μL lysate was mixed with 5 μL Formate Assay Buffer and added to a 96-well plate. 25 μL of the Reaction Mix consisting of 23 μL Formate Assay Buffer, 1 μL Formate Enzyme Mix and 1 μL Formate Substrate Mix was added to the plate containing the lysate. The samples were incubated for 1 h at 37° C. protected from light and the absorbance was measured at 450 nm on a FLUOstar Omega plate reader (BMG Labtech). The data was normalized to protein concentration.

### Extracellular Flux Analysis (SeaHorse)

5×10⁴ NRCs per well were seeded in a XF24 Cell Culture Microplate (SeaHorse Bioscience) and analyzed after 3-4 days. On the assay day, NRCs were cultured in Seahorse XF Media supplemented with 5 mM glucose and extracellular acidification rate (ECAR) and oxygen consumption rate (OCR) were measured simultaneously using the SeaHorse XF24 Flux Analyzer (SeaHorse Bioscience). ECAR and OCAR were measured at basal and after the addition of 1 μM oligomycin and 4.5 μM antimycin A.

### In Vitro Contractility Assay

Contractility assays were performed on neonatal rat cardiomyocytes and analyzed with the Myocyte Calcium & Contractility Recording System (IonOptix). 2.5×10⁵ cardiomyocytes were seeded on square coverslips (Thermo Fisher Scientific) and after 3-4 days placed in a chamber mounted on the stage of an inverted microscope to record contractility. Cells were superfused with a modified tyrode buffer (137 mM NaCl, 5 mM KCl, 15 mM glucose, 1.3 mM MgSO₄, 1.2 mM NaH₂PO₄, 20 mM HEPES, 1 mM CaCl₂, pH 7.4) and field stimulated at a frequency of 1 Hertz (Hz). Contractility was recorded and analyzed using the IonWizard software (version 6.2).

### Metabolomics

4×10⁵ NRCs were cultured per 3 cm dish. The whole cell culture plates were snap frozen in liquid nitrogen after the cells were washed with 75 mM Ammonium carbonate (Sigma), adjusted to pH 7.4 with acetic acid. The metabolites were extracted with cold extraction buffer (−20° C.) containing acetonitrile:methanol:water in a 40:40:20 ratio. Untargeted analysis of metabolites by flow injection-time-of flight mass spectrometry as previously described (Fuhrer et al. Anal Chem 83, 7074-7080 (2011).). Data was processed and analyzed with Matlab. Metabolomics analysis performed by Metabolon (FIG. 7d) was performed as previously described (Cimen et al. Sci Transl Med 8, 358ra126 (2016).).

### Bioinformatic Analysis

In silico promoter analyses were performed using MatInspector (Genomatix). Sequence alignments were performed with BLAST alignment (http://www.ncbi.nlm.nih.gov/blast). shRNAs were designed by proprietary algorithm (Targeted Transgenesis), or computationally predicted using pSico Oligomaker (MIT, version 1.5), BLOCK-iT RNAi Designer (Life Technologies). To predict miR27b targets the miRWalk 2.0 database (http://mirwalk.uni-hd.de) which includes many databases such as TargetScan, miRanda and RNA22 was used (Dweep et al. J Biomed Inform 44, 839-847 (2011).). Confocal images were quantified using Image J (version 1.47) (Schneider et al. Nat Methods 9, 671-675 (2012)).

### Statistical Analysis

For statistical analyses in FIGS. 1a and 1 b, and FIG. 2a the Pearson correlation coefficient was calculated according to the standard formula. Two-tailed unpaired Student's t-tests (Excel) or one-way ANOVA analyses followed by either a Dunnett's multiple comparison post-test (multiple comparisons to a single control) or Bonferroni correction (multiple comparisons between different groups) were used as indicated in the respective figure legends. For statistical analysis of survival curves, the inventors performed log-rank (Mantel-Cox) test. A p value of less than 0.05 was considered statistically significant. No statistical methods were used to predetermine sample size.

1. An agent for use in prevention or treatment of hypertrophic heart disease, wherein the agent is selected from
a. a non-agonist biopolymer ligand specifically binding to MTHFD1L;
b. a nucleic acid capable of specifically suppressing expression of MTHFD1L,
wherein the non-agonist biopolymer ligand is an antibody, an antibody fragment, an antibody-like molecule or an aptamer.

2. The agent according to claim 1 for use in prevention or treatment of hypertrophic heart disease, wherein the non-agonist biopolymer ligand is a human antibody or a humanized antibody.

3. The agent according to claim 1 for use in prevention or treatment of hypertrophic heart disease, wherein the binding of the non-agonist biopolymer ligand to MTHFD1L is characterized by a K_{D} of smaller than (<) 10⁻⁷, particularly K_{D}<10⁻⁸, more particularly K_{D}<10⁻⁹.

4. The agent for use in prevention or treatment of hypertrophic heart disease according to claim 1, wherein the nucleic acid capable of specifically suppressing expression of MTHFD1L is a small-interference RNA (siRNA) or an antisense oligonucleotide.

5. A nucleic acid molecule encoding the agent according to claim 1 for use in prevention or treatment of hypertrophic heart disease.

6. The nucleic acid molecule for use in treatment of hypertrophic heart disease according to claim 5, wherein the nucleic acid molecule is a DNA molecule or an RNA molecule.

7. A nucleic acid expression vector comprising the nucleic acid molecule of claim 5 for use in prevention or treatment of hypertrophic heart disease.

8. The nucleic acid expression vector for use in prevention or treatment of hypertrophic heart disease according to claim 7, wherein the expression vector is selected from:
a. a nucleic expression construct selected from a DNA plasmid, a double stranded linear DNA, and a single stranded RNA, wherein optionally said nucleic acid expression construct is encapsulated in a lipid vesicle, and
b. a viral vector, particularly a lentiviral vector, a herpes viral vector, an adenoviral vector and an adeno-associated viral vector.

9. The agent according to claim 1 for use in prevention or treatment of hypertrophic heart disease, wherein the hypertrophic heart disease is ischemic heart disease or hypertrophic heart disease associated with an elevated risk of infarction.

10. The agent according to claim 1 for use in prevention or treatment of hypertrophic heart disease, wherein the hypertrophic heart disease indication is classified as restrictive cardiomyopathy, pulmonary heart disease, coronary artery disease, renal artery stenosis, aortic stenosis or aneurysm, peripheral arterial disease, hypertensive heart disease, congenital heart disease, vascular disease, valvular heart disease.

11. The agent according to claim 1 for use in prevention or treatment of hypertrophic heart disease, wherein the hypertrophic heart disease is caused by or associated with hypoxia, ischemia, hypertension, stenosis, aneurysm or blockage of major blood vessels, embolisms and thrombosis, or stressors leading to cardiac pressure and/or volume overload.

12. A pharmaceutical composition for use in prevention or treatment of hypertrophic heart disease in a patient, comprising the agent according to claim 1, and a pharmaceutically acceptable carrier, particularly formulated as an administration form for parenteral administration, more particularly for intravenous administration.

13. A method for identifying an MTHFD1L inhibitor, said method comprising the steps of
contacting MTHFD1L with a small molecule,
measuring the activity of MTHFD1L for said small molecule, and
selecting a small molecule as an MTHFD1L inhibitor, wherein enzymatic activity of MTHFD1L is diminished by at least 80%, particularly by at least 90%.


SEQUENCE LISTING (135 sequences)

SEQ ID NO: 1 - DNA, 55 bp, Artificial Sequence
  (Sense)
tgaatggtgtcagagaatttttcaagagaaaattctctgacaccattcttttttc

SEQ ID NO: 2 - DNA, 59 bp, Artificial Sequence
  (antisense)
tcgagaaaaaagaatggtgtcagagaattttctcttgaaaaattctctgacaccattca

SEQ ID NO: 3 - DNA, 48 bp, Artificial Sequence
  (sense)
gcttcgagaggcagacattgtctcgagacaatgtctgcctctcgaagc

SEQ ID NO: 4 - DNA, 21 bp, Artificial Sequence
  primer
tctccatgcctctaacactcg

SEQ ID NO: 5 - DNA, 21 bp, Artificial Sequence
  primer
ccaggtcaacagacgtgtcag

SEQ ID NO: 6 - DNA, 21 bp, Artificial Sequence
  primer
ggttcatcctgccagagacta

SEQ ID NO: 7 - DNA, 21 bp, Artificial Sequence
  primer
aatccctcatcgaactggacg

SEQ ID NO: 8 - DNA, 21 bp, Artificial Sequence
  priemr
ccaggagactgaagtccatca

SEQ ID NO: 9 - DNA, 21 bp, Artificial Sequence
  primer
agaacctgtcccatacactcg

SEQ ID NO: 10 - DNA, 20 bp, Artificial Sequence
  primer
tgcttcaggcgcgtacatac

SEQ ID NO: 11 - DNA, 19 bp, Artificial Sequence
  primer
cacttgcttgacgttggca

SEQ ID NO: 12 - DNA, 21 bp, Artificial Sequence
  primer
caggctggactcagctacatc

SEQ ID NO: 13 - DNA, 21 bp, Artificial Sequence
  primer
gttcgctttgaactcggtctt

SEQ ID NO: 14 - DNA, 23 bp, Artificial Sequence
  primer
agttcctttaccctaagactggt

SEQ ID NO: 15 - DNA, 22 bp, Artificial Sequence
  primer
ttcatgctcgactgctttactt

SEQ ID NO: 16 - DNA, 22 bp, Artificial Sequence
  primer
ggtctaatcaggcctgtgtctg

SEQ ID NO: 17 - DNA, 20 bp, Artificial Sequence
  primer
ccagctcctgatccagccac

SEQ ID NO: 18 - DNA, 20 bp, Artificial Sequence
  primer
cagtggagttgaagcgacca

SEQ ID NO: 19 - DNA, 21 bp, Artificial Sequence
  primer
tgtcgatgtcccttgaaatgg

SEQ ID NO: 20 - DNA, 22 bp, Artificial Sequence
  primer
tctgcatcagtgttatctcggc

SEQ ID NO: 21 - DNA, 21 bp, Artificial Sequence
  primer
caccagaaccagcaactccta

SEQ ID NO: 22 - DNA, 21 bp, Artificial Sequence
  primer
gctgggcgtaaacttgctcta

SEQ ID NO: 23 - DNA, 21 bp, Artificial Sequence
  primer
cagacagactagccaacctgg

SEQ ID NO: 24 - DNA, 22 bp, Artificial Sequence
  primer
gttcaggtctctccactcatca

SEQ ID NO: 25 - DNA, 22 bp, Artificial Sequence
  primer
cggggttttaggtaactgtagc

SEQ ID NO: 26 - DNA, 21 bp, Artificial Sequence
  primer
tattggcccagagtatcagca

SEQ ID NO: 27 - DNA, 23 bp, Artificial Sequence
  primer
ggggtttgtcgatgacttcaaat

SEQ ID NO: 28 - DNA, 21 bp, Artificial Sequence
  primer
tgccgagctggataatgatgc

SEQ ID NO: 29 - DNA, 21 bp, Artificial Sequence
  primer
accatgctaatccccgagatg

SEQ ID NO: 30 - DNA, 19 bp, Artificial Sequence
  primer
gagaaggcaccgtcgatgg

SEQ ID NO: 31 - DNA, 23 bp, Artificial Sequence
  primer
acactctgaatagctgtagggat

SEQ ID NO: 32 - DNA, 20 bp, Artificial Sequence
  primer
tctcgacaggttcggagctt

SEQ ID NO: 33 - DNA, 21 bp, Artificial Sequence
  primer
agagtacagggcggttgcata

SEQ ID NO: 34 - DNA, 23 bp, Artificial Sequence
  primer
tctgggaatggttgaatacagtg

SEQ ID NO: 35 - DNA, 20 bp, Artificial Sequence
  primer
ctccacaccgaagtagccac

SEQ ID NO: 36 - DNA, 21 bp, Artificial Sequence
  primer
tggcatgagtgagttggtgac

SEQ ID NO: 37 - DNA, 22 bp, Artificial Sequence
  primer
tgcaacagcgttcttttcttct

SEQ ID NO: 38 - DNA, 21 bp, Artificial Sequence
  primer
gcctcattcattaccccaaca

SEQ ID NO: 39 - DNA, 20 bp, Artificial Sequence
  primer
gacggttgttgattaggcgt

SEQ ID NO: 40 - DNA, 20 bp, Artificial Sequence
  primer
cacaaacattcccactggca

SEQ ID NO: 41 - DNA, 21 bp, Artificial Sequence
  primer
gttggggtaatgaatgaggca

SEQ ID NO: 42 - DNA, 22 bp, Artificial Sequence
  primer
tgatgcttgtcaaatgctcagc

SEQ ID NO: 43 - DNA, 21 bp, Artificial Sequence
  primer
aggtcctcctgtactgctcat

SEQ ID NO: 44 - DNA, 21 bp, Artificial Sequence
  primer
gccttcaccattcatgtggat

SEQ ID NO: 45 - DNA, 21 bp, Artificial Sequence
  primer
ttgctccgggtaaagagacag

SEQ ID NO: 46 - DNA, 21 bp, Artificial Sequence
  primer
gcgtaccctgacaccaatctc

SEQ ID NO: 47 - DNA, 21 bp, Artificial Sequence
  primer
ctcctcttcgcacttctgctc

SEQ ID NO: 48 - DNA, 20 bp, Artificial Sequence
  primer
gtggctccgacctttcagtc

SEQ ID NO: 49 - DNA, 22 bp, Artificial Sequence
  primer
cacagtcttgtcaatcttggca

SEQ ID NO: 50 - DNA, 21 bp, Artificial Sequence
  primer
agaaggtacttacggtgtggt

SEQ ID NO: 51 - DNA, 22 bp, Artificial Sequence
  primer
gagagatttcccgaattgcagt

SEQ ID NO: 52 - DNA, 19 bp, Artificial Sequence
  primer
tcccgtcagagggatgagc

SEQ ID NO: 53 - DNA, 20 bp, Artificial Sequence
  primer
gctgggagtttctcaggagg

SEQ ID NO: 54 - DNA, 23 bp, Artificial Sequence
  primer
cccttaacaagctgaatctgaca

SEQ ID NO: 55 - DNA, 22 bp, Artificial Sequence
  primer
ggcatcactttgctaaagcagt

SEQ ID NO: 56 - DNA, 22 bp, Artificial Sequence
  primer
cagttcggctataacactggtg

SEQ ID NO: 57 - DNA, 19 bp, Artificial Sequence
  primer
gcccccgacagagaagatg

SEQ ID NO: 58 - DNA, 19 bp, Artificial Sequence
  primer
cggtgtgcggctatcagac

SEQ ID NO: 59 - DNA, 21 bp, Artificial Sequence
  primer
cagggcataccagtaactcca

SEQ ID NO: 60 - DNA, 20 bp, Artificial Sequence
  primer
tgctcatcagttgccacttc

SEQ ID NO: 61 - DNA, 20 bp, Artificial Sequence
  primer
cggcatccagaagttttctc

SEQ ID NO: 62 - DNA, 21 bp, Artificial Sequence
  primer
tcagtcaacgggggacataaa

SEQ ID NO: 63 - DNA, 21 bp, Artificial Sequence
  primer
ggggctgtactgcttaaccag

SEQ ID NO: 64 - DNA, 22 bp, Artificial Sequence
  primer
tgtctccagcaaagactactgt

SEQ ID NO: 65 - DNA, 23 bp, Artificial Sequence
  primer
gactgtacttgacaatgttggga

SEQ ID NO: 66 - DNA, 22 bp, Artificial Sequence
  primer
gcggagaggatgagatcataga

SEQ ID NO: 67 - DNA, 19 bp, Artificial Sequence
  primer
gtcaccccgtccacatctt

SEQ ID NO: 68 - DNA, 16 bp, Artificial Sequence
  primer
agatgaggtcatgccc

SEQ ID NO: 69 - DNA, 16 bp, Artificial Sequence
  primer
aagctgttgcagccta

SEQ ID NO: 70 - DNA, 24 bp, Artificial Sequence
  primer
ccagtctccagagcaattcaagat

SEQ ID NO: 71 - DNA, 24 bp, Artificial Sequence
  primer
gctaattcacaaaggactcgaggt

SEQ ID NO: 72 - DNA, 21 bp, Artificial Sequence
  primer
ggacttcgggtcagtgaatgc

SEQ ID NO: 73 - DNA, 22 bp, Artificial Sequence
  primer
cgcagaaacataaacgaggtct

SEQ ID NO: 74 - DNA, 20 bp, Artificial Sequence
  primer
tgcagagcttagctgattgg

SEQ ID NO: 75 - DNA, 21 bp, Artificial Sequence
  primer
ccttctcttcaggtgcagaac

SEQ ID NO: 76 - DNA, 21 bp, Artificial Sequence
  primer
gcgggactcaagtacaactcc

SEQ ID NO: 77 - DNA, 22 bp, Artificial Sequence
  primer
cattccgataatggaagtgcca

SEQ ID NO: 78 - DNA, 23 bp, Artificial Sequence
  primer
gcacatagagagaatgagcttcc

SEQ ID NO: 79 - DNA, 19 bp, Artificial Sequence
  primer
ctccgctctgaacaaggct

SEQ ID NO: 80 - DNA, 22 bp, Artificial Sequence
  primer (human)
ctgtaggcaggaaaataatagg

SEQ ID NO: 81 - DNA, 20 bp, Artificial Sequence
  primer (human)
gggtgagagagatggagacc

SEQ ID NO: 82 - DNA, 19 bp, Artificial Sequence
  primer (human)
cctgtcagggactatgcgg

SEQ ID NO: 83 - DNA, 21 bp, Artificial Sequence
  primer (human)
tccttactgtgctctcaccca

SEQ ID NO: 84 - DNA, 21 bp, Artificial Sequence
  primer (human)
ggtagcggcagcaaaatatgc

SEQ ID NO: 85 - DNA, 21 bp, Artificial Sequence
  primer
ccacacagtcctcgatctgag

SEQ ID NO: 86 - DNA, 19 bp, Artificial Sequence
  primer (human)
tcccacgcaggtgttcttc

SEQ ID NO: 87 - DNA, 20 bp, Artificial Sequence
  primer (human)
ggaaccgctgctcacaaagt

SEQ ID NO: 88 - DNA, 19 bp, Artificial Sequence
  primer (human)
gtggcctactggagacagg

SEQ ID NO: 89 - DNA, 22 bp, Artificial Sequence
  primer (human)
ggagtatcggatgtagctgagt

SEQ ID NO: 90 - DNA, 19 bp, Artificial Sequence
  primer (human)
aggtccaggggtattgcag

SEQ ID NO: 91 - DNA, 22 bp, Artificial Sequence
  primer (human)
tcctcagggatcagtccataac

SEQ ID NO: 92 - DNA, 20 bp, Artificial Sequence
  primer (human)
ccaggaacccgtctctcaag

SEQ ID NO: 93 - DNA, 21 bp, Artificial Sequence
  primer (human)
ggaaggcgaccatcaaacaga

SEQ ID NO: 94 - DNA, 20 bp, Artificial Sequence
  primer (human)
ccctccttggtcaagagcac

SEQ ID NO: 95 - DNA, 21 bp, Artificial Sequence
  primer (human)
gtatctccggtcgtttcagca

SEQ ID NO: 96 - DNA, 22 bp, Artificial Sequence
  primer (human)
ccagagttgcatacagaccaat

SEQ ID NO: 97 - DNA, 23 bp, Artificial Sequence
  primer (human)
cccattaaataccgtagagccct

SEQ ID NO: 98 - DNA, 21 bp, Artificial Sequence
  primer (human)
gctgggcgaaaacttgctcta

SEQ ID NO: 99 - DNA, 21 bp, Artificial Sequence
  primer (human)
ccagtcgatagctggtggatt

SEQ ID NO: 100 - DNA, 21 bp, Artificial Sequence
  primer (human)
caggtctctccgctcatcaag

SEQ ID NO: 101 - DNA, 22 bp, Artificial Sequence
  primer (human)
gcccgaggttttaggtaattgt

SEQ ID NO: 102 - DNA, 21 bp, Artificial Sequence
  primer (human)
ggaggacctgttgatgctagt

SEQ ID NO: 103 - DNA, 23 bp, Artificial Sequence
  primer (human)
tggggtttttcgatgacttcaaa

SEQ ID NO: 104 - DNA, 20 bp, Artificial Sequence
  primer (human)
cggagcgtttcaaagaggtt

SEQ ID NO: 105 - DNA, 20 bp, Artificial Sequence
  primer (human)
atgccagcaccatggtaatc

SEQ ID NO: 106 - DNA, 21 bp, Artificial Sequence
  primer (human)
atggcccaatttgtccgtaac

SEQ ID NO: 107 - DNA, 21 bp, Artificial Sequence
  primer (human)
tggcgtagtaccaaaatgtgg

SEQ ID NO: 108 - DNA, 22 bp, Artificial Sequence
  primer (human)
cgcattttggtactacaccacg

SEQ ID NO: 109 - DNA, 23 bp, Artificial Sequence
  primer (human)
tgcctgtgatctctctgacataa

SEQ ID NO: 110 - DNA, 21 bp, Artificial Sequence
  primer (human)
attgaaggtcgctatgccaca

SEQ ID NO: 111 - DNA, 23 bp, Artificial Sequence
  primer (human)
gcttttcactttaatggaacgct

SEQ ID NO: 112 - DNA, 21 bp, Artificial Sequence
  primer (human)
agcagttgtgtggcgtaaaga

SEQ ID NO: 113 - DNA, 23 bp, Artificial Sequence
  primer (human)
ctgatgcgatcataatccacctt

SEQ ID NO: 114 - DNA, 21 bp, Artificial Sequence
  primer (human)
cctggcgtcgtgattagtgat

SEQ ID NO: 115 - DNA, 22 bp, Artificial Sequence
  primer (human)
agacgttcagtcctgtccataa

SEQ ID NO: 116 - DNA, 20 bp, Artificial Sequence
  primer (human)
cacctacaccccttatcccc

SEQ ID NO: 117 - DNA, 20 bp, Artificial Sequence
  primer (human)
gcctgcagtaatgttagcgg

SEQ ID NO: 118 - DNA, 20 bp, Artificial Sequence
  primer (human)
cacctacctccctcaccaaa

SEQ ID NO: 119 - DNA, 21 bp, Artificial Sequence
  primer (human)
ggcaatgaatgaagcgaacag

SEQ ID NO: 120 - DNA, 20 bp, Artificial Sequence
  primer (human)
gaggtcccgatgcttgtcag

SEQ ID NO: 121 - DNA, 21 bp, Artificial Sequence
  primer (human)
gttagcagccctagcactgtc

SEQ ID NO: 122 - DNA, 19 bp, Artificial Sequence
  primer (human)
cgctggcggtactgaagtc

SEQ ID NO: 123 - DNA, 21 bp, Artificial Sequence
  primer (human)
gaggaacggtgacatgctcat

SEQ ID NO: 124 - DNA, 20 bp, Artificial Sequence
  primer (human)
gctgcgaagtggaaaccatc

SEQ ID NO: 125 - DNA, 22 bp, Artificial Sequence
  primer (human)
cctccttctgcacacatttgaa

SEQ ID NO: 126 - DNA, 21 bp, Artificial Sequence
  primer (human)
gccagccttgggacaataatg

SEQ ID NO: 127 - DNA, 20 bp, Artificial Sequence
  primer (human)
cttgcacgttgagtttgggt

SEQ ID NO: 128 - DNA, 22 bp, Artificial Sequence
  primer (human)
ggatgtgcttatgcaggattcc

SEQ ID NO: 129 - DNA, 23 bp, Artificial Sequence
  primer (human)
catgtactgaccaggagggatag

SEQ ID NO: 130 - DNA, 21 bp, Artificial Sequence
  primer (human)
caacgcagacctgatggattt

SEQ ID NO: 131 - DNA, 19 bp, Artificial Sequence
  primer (human)
agcccccgcttcttcattc

SEQ ID NO: 132 - DNA, 20 bp, Artificial Sequence
  primer (human)
tggaaacgtccgggttacag

SEQ ID NO: 133 - DNA, 20 bp, Artificial Sequence
  primer (human)
ctgatccggtccatcttcct

SEQ ID NO: 134 - DNA, 22 bp, Artificial Sequence
  primer
gcatgctgatttgtgacttgag

SEQ ID NO: 135 - DNA, 21 bp, Artificial Sequence
  primer
cctctgttctccaaactgcag